(19) United States
(12) Patent Application Publication
(10) Pub. No.: US 2023/0393125 A1
(43) Pub. Date: Dec. 7, 2023

(54) METHOD FOR DETERMINING THE FREE ANTIGEN OF AN ANTIBODY IN A SAMPLE

(71) Applicant: Hoffmann-La Roche Inc., Little Falls, NJ (US)

(72) Inventors: Gregor JORDAN, Gröbenzell (DE); Martin SCHAEFER, Muenchen (DE); Maria VIERT, Penzberg (DE)

(73) Assignee: Hoffmann-La Roche Inc., Little Falls, NJ (US)

(21) Appl. No.: 18/067,567

(22) Filed: Dec. 16, 2022

Related U.S. Application Data
(63) Continuation of application No. PCT/EP2021/065880, filed on Jun. 14, 2021.

(30) Foreign Application Priority Data
Jun. 16, 2020 (EP) 20180205.5

(51) Int. Cl. G01N 33/543; G01N 33/68

(52) U.S. Cl. CPC G01N 33/54306; G01N 2333/4716; G01N 2333/521; G01N 33/6863

(57) ABSTRACT

Herein is reported a method for determining free antigen of an antibody in an undiluted serum sample comprising the steps of a) applying the undiluted sample to a solid phase on which a capture antibody has been immobilized to form a capture antibody-antigen complex, wherein the capture antibody competes with the antibody for binding to a first epitope on the antigen, b) applying to the solid phase a tracer antibody to form a capture antibody-antigen-tracer antibody complex, wherein the tracer antibody specifically binds to a second epitope on the antigen, wherein the epitope of the tracer antibody is not overlapping with the epitope of the capture antibody on the antigen, and c) determining the free antigen of the antibody by determining the tracer antibody in the capture antibody-antigen-tracer antibody complex.

The current invention is in the field of pharmacokinetics. More specifically, herein is reported an assay for the determination of the free antigen of a therapeutic antibody in a sample, especially in the presence of the therapeutic antibody, at high serum concentrations.

### BACKGROUND OF THE INVENTION

Lee, J. W., et al. (AAPS J. 13 (2011) 99-110) report that the predominant driver of bioanalysis in supporting drug development is the intended use of the data. Reliable methodologies for measurements of mAb and its antigen ligand (L) in circulation are crucial for the assessment of exposure—response relationships in support of efficacy and safety evaluations, and dose selection. Ligand-binding assays (LBA) are widely used for the analysis of protein biotherapeutics and antigen ligands (L) to support pharmacokinetics/pharmacodynamics (PK/PD) and safety assessments. For monoclonal antibody drugs (mAb), in particular, which non-covalently bind to L, multiple forms of mAb and L can exist in vivo, including free mAb, free L, and mono- and/or bivalent complexes of mAb and L. Given the complexity of the dynamic binding equilibrium occurring in the body after dosing and multiple sources of perturbation of the equilibrium during bioanalysis, it is clear that ex vivo quantification of the forms of interest (free, bound, or total mAb and L) may differ from the actual ones in vivo. LBA reagents and assay formats can be designed in principle to measure the total or free forms of mAb and L. However, confirmation of the forms being measured under the specified conditions can be technically challenging.

Generally, commercially available assays for the detection of analytes are performed with a minimum required dilution (MRD) of 1:2 or more (Gyrolab's affinity flow-through format). The drawback of applying dilutions is amongst other things that complexes in the sample, e.g. antibody-antigen-complexes, are forced to dissociate by the applied solution. Thereby the assay result is no longer reflecting the true situation in the sample.

WO 2018/075758 reported a method of quantitating free (unbound) human C5 complement protein (C5) from a sample comprising: binding biotinylated anti-C5 capture antibody to streptavidin-coated particles; capturing the free (unbound) C5 in the sample; detecting the captured free C5; and quantitating the captured free C5 using laser-induced fluorescence detection.

Takashi, I., et al. report increased cerebrospinal fluid complement C5 levels in major depressive disorder and schizophrenia” (Biochem. Biophys. Res. Commun. 497 (2018) 683-688.

Roth, A., et al. reported the complement C5 inhibitor Crovalimab in paroxysmal nocturnal hemoglobinuria (Blood 135 (2020) 912-920).

Haringman, J., et al. reported a randomized controlled trial with an anti-CCL2 (anti-monocyte chemotactic protein 1) monoclonal antibody in patients with rheumatoid arthritis (Arth. Rheum. 54 (2006) 2387-2392.

Thus, there is the need for assays especially for the determination of free antigen, i.e. non-complexed antigen, in the presence of the complexed antigen and antibody specifically binding to the antigen.

### SUMMARY OF THE INVENTION

Herein is reported a method for the detection of the presence and for the determination of the amount of free antigen, i.e. the antigen in non-complexed form, of a (therapeutic) antibody in a serum sample, whereby the serum sample comprises the antigen, the (therapeutic) antibody and complexed antigen, i.e. the antigen in an (therapeutic) antibody-antigen-complex. The antigen can be specifically bound by the therapeutic antibody, such as, e.g., by a first binding specificity of the (multispecific, therapeutic) antibody.

The current invention is based, at least in part, on the finding that free antigen determination in qualitative and quantitative form can be done without sample dilution, i.e. in 100% serum. Thereby complex dissociation and falsification of the determination can be prevented.

The current invention is based, at least in part, on the finding that by omitting sample dilution steps prior to analysis the falsification of the result in the determination of free antigen can be reduced or even prevented. This is especially true in cases wherein the antibody-antigen-complex has a short half-life, i.e. is quite instable. This is especially the case when the complex half-life is less than 600 seconds, less than 300 seconds, and especially less than 100 seconds. Without being bound by this theory it is assumed that by the dilution due to the kinetic properties of the complex, i.e. the short half-life of the complex or the low affinity of the (monovalent) antibody (binding site) to the antigen, complexes present in the sample tend to/are forced to dissociate during/while the assay is performed. Thereby the amount of free antigen is increased resulting in a falsification of the assay result.

The current invention is based, at least in part, on the finding that by using as capture and/or tracer antibody an antibody binding to the same or an overlapping epitope as that of the therapeutic antibody in combination with short incubation times in a bridging assay format the displacement of the (therapeutic) antibody in antigen-antibody complexes can be prevented and the falsification of the result in the determination of free antigen can be reduced or even prevented. Without being bound by this theory it is assumed that by using capture and/or tracer antibodies binding to the same or an overlapping epitope as the therapeutic antibody the exclusion of antigen-(therapeutic) antibody-complexes in the determination of free antigen is achieved. Thereby the amount of free antigen is not increased resulting in a better assay result.

Thus, the current invention comprises at least the following aspects and embodiments:
1. A method for determining free antigen of an antibody in a serum sample, wherein the method comprises the following steps:
   a) applying the sample to a solid phase on which a capture antibody has been immobilized to form a capture antibody-antigen complex,
      wherein the capture antibody competes with the antibody for binding to a first epitope on the antigen,
   b) applying to the solid phase a tracer antibody to form a capture antibody-antigen-tracer antibody complex,
      wherein the tracer antibody specifically binds to a second epitope on the antigen,
      wherein the epitope of the tracer antibody is not overlapping with the epitope of the capture antibody on the antigen,
   c) determining the free antigen of the antibody by determining the tracer antibody in the capture antibody-antigen-tracer antibody complex.
2. The method according to item 1, wherein the sample comprises free antigen, free antibody and antigen-antibody-complexes.
3. The method according to any one of items 1 to 2, wherein the method is an antigen bridging assay.
4. The method according to any one of items 1 to 3, wherein the applying in step a) is under conditions that at most 10% of the antibody bound to the antigen are replaced by the capture antibody or wherein in step a) at most 10% of the antibody bound to the antigen is replaced by the capture antibody.
5. The method according to any one of items 1 to 4, wherein the applying in step a) is under conditions that at most 5% of the antibody bound to the antigen are replaced by the capture antibody or wherein in step a) at most 5% of the antibody bound to the antigen is replaced by the capture antibody.
6. The method according to any one of items 1 to 5, wherein the applying in step a) is under conditions that at most 1% of the antibody bound to the antigen are replaced by the capture antibody or wherein in step a) at most 1% of the antibody bound to the antigen is replaced by the capture antibody.
7. The method according to any one of items 1 to 6, wherein the applying in step a) is under conditions that substantially no antibody bound to the antigen is replaced by the capture antibody.
8. The method according to any one of items 1 to 7, wherein the serum sample is an undiluted serum sample.
9. The method according to any one of items 1 to 7, wherein the sample comprises about 100% serum.
10. The method according to any one of items 1 to 9, wherein step a) is
   applying the sample to the solid phase on which a capture antibody has been immobilized to form a capture antibody-antigen complex,
   wherein the capture antibody competes with the antibody for binding to a first epitope on the antigen,
   wherein the sample is incubated with the solid phase for/is removed from the solid phase after 300 seconds or less.
11. The method according to any one of items 1 to 10, wherein the sample is incubated with the solid phase for/is removed from the solid phase after 240 seconds or less
12. The method according to any one of items 1 to 11, wherein the sample is incubated with the solid phase for/is removed from the solid phase after 100 seconds or less.
13. The method according to any one of items 1 to 12, wherein the sample is incubated with the solid phase for/is removed from the solid phase after, more preferably for/after 10 seconds or less.
14. The method according to any one of items 1 to 13, wherein the sample is incubated with the solid phase for/is removed from the solid phase after 2 seconds or less.
15. The method according to any one of items 1 to 14, wherein the sample is incubated with the solid phase for/is removed from the solid phase after 1 second or less.
16. The method according to any one of items 1 to 15, wherein the stability/half-life of the complex of the antigen binding site of the antibody specifically binding to the first epitope on the antigen and the antigen (monovalent, non-avid interaction) is 200 seconds or less.
17. The method according to any one of items 1 to 16, wherein the stability/half-life of the complex of the antigen binding site of the antibody specifically binding to the first epitope on the antigen and the antigen (monovalent, non-avid interaction) is 100 seconds or less.
18. The method according to any one of items 1 to 17, wherein the antibody is a bispecific antibody; wherein the bispecific antibody comprises a first antigen-binding site that (specifically) binds to the first epitope on the antigen and a second different antigen-binding site that (specifically) binds the second epitope on the antigen, wherein the tracer antibody competes with the bispecific antibody for binding to the second epitope on the antigen.
19. The method according to item 18, wherein the stability/half-life of the complex of the antigen binding site of the bispecific antibody specifically binding to the second epitope on the antigen and the antigen (monovalent, non-avid interaction) is smaller than that of the complex of the first binding site of the bispecific antibody specifically binding to the first epitope on the antigen and the antigen (monovalent, non-avid interaction).
20. The method according to any one of items 18 to 19, wherein the stability/half-life of the complex of the antigen binding site of the bispecific antibody specifically binding to the second epitope on the antigen and the antigen (monovalent, non-avid interaction) is 100 seconds or less.
21. The method according to any one of items 18 to 20, wherein the stability/half-life of the complex of the antigen binding site of the bispecific antibody specifically binding to the second epitope on the antigen and the antigen (monovalent, non-avid interaction) is 20 seconds or less.
22. The method according to any one of items 1 to 21, wherein the capture antibody and the tracer antibody is a non-human, non-humanized antibody.
23. The method according to any one of items 1 to 22, wherein the method is an enzyme-linked immunosorbent assay and the sample is incubated with the solid phase for/is removed from the solid phase after 180 to 240 seconds.
24. The method according to any one of items 1 to 23, wherein the tracer antibody is incubated with the capture antibody-antigen complex for less than 1200 seconds.
25. The method according to any one of items 1 to 22, wherein the method is a nanoliter-scale, microfluidic, affinity flow-through format with laser-induced fluorescence detection and the sample is incubated with the solid phase for/is removed from the solid phase after 2 seconds or 1 second or less.
26. The method according to any one of items 1 to 22 and 25, wherein the tracer antibody is incubated with the capture antibody-antigen complex for less than 2 seconds or less than 1 second.
27. The method according to any one of items 1 to 26, wherein the antibody is a therapeutic antibody.
28. The method according to any one of items 1 to 27, wherein the antibody is a multispecific antibody.
29. The method according to any one of items 1 to 28, wherein the antibody is a bispecific antibody.
30. The method according to any one of items 1 to 29, wherein the antibody is a bispecific antibody with a first binding site specifically binding to a first epitope on the antigen and a second binding site specifically binding to a second epitope on the antigen.
31. The method according to any one of items 1 to 30, wherein the method is for determining the amount of free antigen, and step c) is determining the amount of free antigen of the antibody in the sample by determining the amount of the tracer antibody in the capture antibody-antigen-tracer antibody complex.
32. The method according to any one of items 1 to 31, wherein the determining of the free antigen is by incubating the capture antibody-antigen-tracer antibody complex with a detection antibody conjugated to a detectable label and determining the signal produced by the detectable label.
33. The method according to any one of items 31 to 32, wherein the determining the amount of free antigen is by correlating the signal produced by the detectable label of the detection antibody bound to the capture antibody-antigen-tracer antibody complex with the amount of the free antigen using a calibration curve.
34. The method according to any one of items 1 to 33, wherein the antigen is human CCL2.
35. The method according to any one of items 1 to 34, wherein the (therapeutic) antibody is an antibody specifically binding to human CCL2 as described herein.
36. The method according to any one of items 1 to 33, wherein the antigen is human C5.
37. The method according to any one of items 1 to 33 and 36, wherein the (therapeutic) antibody is an antibody specifically binding to human C5 as described herein.
38. The method according to any one of items 1 to 37, wherein the serum is human serum.
39. An in vitro method for the determination of the presence and/or the amount of an antigen of a bispecific antibody/that can be specifically bound by a bispecific antibody in a sample, whereby the antigen to be detected can be specifically bound by at least a first binding specificity of the bispecific antibody, and whereby the antigen is free antigen, comprising the step(s) as disclosed herein.
40. The method according to item 39, wherein the sample is an undiluted serum sample.

### DETAILED DESCRIPTION OF THE INVENTION

Herein is reported an in vitro method for the detection of free antigen of multispecific binders, such as bispecific antibodies/drugs, in pre-clinical and clinical samples in the presence of the multispecific binder.

### Definitions

The terms “therapeutic antibody” and “drug” are used interchangeably herein. These terms are used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

In certain embodiments of the invention, the drug is a multispecific antibody, e.g. a bispecific antibody. Multispecific antibodies are monoclonal antibodies that have binding specificities for at least two different antigens. In certain embodiments of the invention, one of the binding specificities is for a first antigen and the other is for a different second antigen. In certain embodiments of the invention, bispecific antibodies may bind to two different epitopes of the same antigen. Bispecific antibodies can be prepared as full-length antibodies or antibody fragments. In certain embodiments of the invention, the antibody is a bispecific antibody, which specifically binds to a first and a second antigen. In certain embodiments of the invention, the bispecific antibody has i) a first binding specificity that specifically binds to a first antigen or a first epitope on an antigen, and ii) a second binding specificity that specifically binds to a second antigen or a second epitope on the (same) antigen. In certain embodiments of the invention, the second epitope on the same antigen is a non-overlapping epitope. In certain embodiments of the invention, the antibody is a bispecific, bivalent antibody. In one preferred embodiment, the antibody is a monoclonal, bispecific, bivalent antibody.

Multispecific antibodies are described in WO 2009/080251, WO 2009/080252, WO 2009/080253, WO 2009/080254, WO 2010/112193, WO 2010/115589, WO 2010/136172, WO 2010/145792, or WO 2010/145793.

The terms “anti-C5 antibody” and “an antibody that (specifically) binds to C5” refer to an antibody that is capable of binding C5 with sufficient affinity such that the antibody is useful as a diagnostic and/or therapeutic agent in targeting C5. In certain embodiments of the invention, the extent of binding of an anti-C5 antibody to an unrelated, non-C5 protein is less than about 10% of the binding of the antibody to C5. In certain embodiments of the invention, an anti-C5 antibody binds to an epitope of C5 that is conserved among C5 from different species. In one preferred embodiment, C5 is human C5.

The term “C5”, as used herein, encompasses any native C5 from any vertebrate source, including mammals such as primates (e.g., humans and monkeys) and rodents (e.g., mice and rats). Unless otherwise indicated, the term “C5” refers to a human C5 protein having the amino acid sequence shown in SEQ ID NO: 30 and containing the beta chain sequence shown in SEQ ID NO: 31. The term encompasses “full-length”, unprocessed C5 as well as any form of C5 that results from processing in the cell. The term also encompasses naturally occurring variants of C5, e.g., splice variants or allelic variants. The amino acid sequence of an exemplary human C5 is shown in SEQ ID NO: 30 (“wild-type” or “wt” C5). The amino acid sequence of an exemplary beta chain of human C5 is shown in SEQ ID NO: 31. The amino acid sequences of exemplary MG1, MG2 and MG1-MG2 domains of the beta chain of human C5 are shown in SEQ ID NO: 32, 33, and 34, respectively. The amino acid sequences of exemplary cynomolgus monkey and murine C5 are shown in SEQ ID NO: 35 and 96, respectively. Amino acid residues 1-19 of SEQ ID NOs: 30, 31, 34, and 96 correspond to a signal sequence that is removed during processing in the cell and is thus missing from the corresponding exemplary amino acid sequence.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The principles of different immunoassays are described, for example, by Hage, D. S. (Anal. Chem. 71 (1999) 294R-304R). Lu, B., et al. (Analyst 121 (1996) 29R-32R) report the orientated immobilization of antibodies for the use in immunoassays. Avidin-biotin-mediated immunoassays are reported, for example, by Wilchek, M., and Bayer, E. A., in Methods Enzymol. 184 (1990) 467-469.

Monoclonal antibodies and their constant domains contain a number of reactive amino acid side chains for conjugating to a member of a binding pair, such as a polypeptide/protein, a polymer (e.g. PEG, cellulose or polystyrol), or an enzyme. Chemical reactive groups of amino acids are, for example, amino groups (lysins, alpha-amino groups), thiol groups (cystins, cysteines, and methionins), carboxylic acid groups (aspartic acids, glutamic acids), and sugar-alcoholic groups. Such methods are e.g. described by Aslam M., and Dent, A., in “Bioconjugation”, MacMillan Ref. Ltd. 1999, pages 50-100.

One of the most common reactive groups of antibodies is the aliphatic ε-amine of the amino acid lysine. In general, nearly all antibodies contain abundant lysine. Lysine amines are reasonably good nucleophiles above pH 8.0 (pKa=9.18) and therefore react easily and cleanly with a variety of reagents to form stable bonds.

Amine-reactive reagents react primarily with lysins and the ct-amino groups of proteins. Reactive esters, particularly N-hydroxy-succinimide (NHS) esters, are among the most commonly employed reagents for modification of amine groups. The optimum pH for reaction in an aqueous environment is pH 8.0 to 9.0. Isothiocyanates are amine-modification reagents and form thiourea bonds with proteins. They react with protein amines in aqueous solution (optimally at pH 9.0 to 9.5). Aldehydes react under mild aqueous conditions with aliphatic and aromatic amines, hydrazines, and hydrazides to form an imine intermediate (Schiffs base). A Schiffs base can be selectively reduced with mild or strong reducing agents (such as sodium borohydride or sodium cyanoborohydride) to derive a stable alkyl amine bond. Other reagents that have been used to modify amines are acid anhydrides. For example, diethylenetriaminepentaacetic anhydride (DTPA) is a bifunctional chelating agent that contains two amine-reactive anhydride groups. It can react with N-terminal and ε-amine groups of amino acids to form amide linkages. The anhydride rings open to create multivalent, metal-chelating arms able to bind tightly to metals in a coordination complex.

Another common reactive group in antibodies is the thiol residue from the sulfur-containing amino acid cystine and its reduction product cysteine (or half cystine). Cysteine contains a free thiol group, which is more nucleophilic than amines and is generally the most reactive functional group in a protein. Thiols are generally reactive at neutral pH, and therefore can be coupled to other molecules selectively in the presence of amines. Since free sulfhydryl groups are relatively reactive, proteins with these groups often exist with them in their oxidized form as disulfide groups or disulfide bonds. In such proteins, reduction of the disulfide bonds with a reagent such as dithiotreitol (DTT) is required to generate the reactive free thiol. Thiol-reactive reagents are those that will couple to thiol groups on polypeptides, forming thioether-coupled products. These reagents react rapidly at slight acidic to neutral pH and therefore can be reacted selectively in the presence of amine groups. The literature reports the use of several thiolating crosslinking reagents such as Traut's reagent (2-iminothiolane), succinimidyl (acetylthio) acetate (SATA), and sulfosuccinimidyl 6-[3-(2-pyridyldithio) propionamido] hexanoate (Sulfo-LC-SPDP) to provide efficient ways of introducing multiple sulfhydryl groups via reactive amino groups. Haloacetyl derivatives, e.g. iodoacetamides, form thioether bonds and are also reagents for thiol modification. Further useful reagents are maleimides. The reaction of maleimides with thiol-reactive reagents is essentially the same as with iodoacetamides. Maleimides react rapidly at slight acidic to neutral pH.

Another common reactive group in antibodies are carboxylic acids. Antibodies contain carboxylic acid groups at the C-terminal position and within the side chains of aspartic acid and glutamic acid. The relatively low reactivity of carboxylic acids in water usually makes it difficult to use these groups to selectively modify polypeptides and antibodies. When this is done, the carboxylic acid group is usually converted to a reactive ester by the use of a water-soluble carbodiimide and reacted with a nucleophilic reagent such as an amine, hydrazide, or hydrazine. The amine-containing reagent should be weakly basic in order to react selectively with the activated carboxylic acid in the presence of the more highly basic ε-amines of lysine to form a stable amide bond. Protein crosslinking can occur when the pH is raised above 8.0.

Sodium periodate can be used to oxidize the alcohol part of a sugar within a carbohydrate moiety attached to an antibody to an aldehyde. Each aldehyde group can be reacted with an amine, hydrazide, or hydrazine as described for carboxylic acids. Since the carbohydrate moiety is predominantly found on the crystallizable fragment region (Fc-region) of an antibody, conjugation can be achieved through site-directed modification of the carbohydrate away from the antigen-binding site. A Schiffs base intermediate is formed, which can be reduced to an alkyl amine through the reduction of the intermediate with sodium cyanoborohydride (mild and selective) or sodium borohydride (strong) water-soluble reducing agents.

The conjugation of a tracer and/or capture and/or detection antibody to its conjugation partner can be performed by different methods, such as chemical binding, or binding via a binding pair. The term “conjugation partner” as used herein denotes e.g. solid supports, polypeptides, detectable labels, members of specific binding pairs. In certain embodiments of the invention, the conjugation of the capture and/or tracer and/or detection antibody to its conjugation partner is performed by chemically binding via N-terminal and/or ε-amino groups (lysine), ε-amino groups of different lysins, carboxy-, sulfhydryl-, hydroxyl-, and/or phenolic functional groups of the amino acid backbone of the antibody, and/or sugar alcohol groups of the carbohydrate structure of the antibody. In certain embodiments of the invention, the capture antibody is conjugated to its conjugation partner via a binding pair. In one preferred embodiment the capture antibody is conjugated to biotin and immobilization to a solid support is performed via solid support immobilized avidin or streptavidin. In certain embodiments of the invention, the capture antibody is conjugated to its conjugation partner via a binding pair. In one preferred embodiment, the tracer antibody is conjugated to digoxygenin by a covalent bond as detectable label.

The term “sample” includes, but is not limited to, any quantity of a substance from a living thing or formerly living thing. Such living things include, but are not limited to, humans, mice, monkeys, rats, rabbits, and other animals. In certain embodiments of the invention, the sample is obtained from a monkey, especially a cynomolgus monkey, or a rabbit, or a mouse, or rat, or a human. In one preferred embodiment, the sample is a human sample. Such substances include, but are not limited to, in certain embodiments whole blood, plasma or serum from an individual, which are the most widely used sources of sample in clinical routine.

The term “solid phase” denotes a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase component is distinguished from inert solid surfaces in that a “solid phase” contains at least one moiety on its surface, which is intended to interact with a substance in a sample. A solid phase may be a stationary component, such as a tube, strip, cuvette or microtiter plate, or may be non-stationary components, such as beads and microparticles. A variety of microparticles that allow either non-covalent or covalent attachment of proteins and other substances may be used. Such particles include polymer particles such as polystyrene and poly (methyl methacrylate); gold particles such as gold nanoparticles and gold colloids; and ceramic particles such as silica, glass, and metal oxide particles. See for example Martin, C. R., et al., Analytical Chemistry-News & Features, 70 (1998) 322A-327A, or Butler, J. E., Methods 22 (2000) 4-23.

Chromogens (fluorescent or luminescent groups and dyes), enzymes, NMR-active groups or metal particles, haptens, e.g. digoxygenin, are examples of “detectable labels”. The detectable label can also be a photoactivatable crosslinking group, e.g. an azido or an azirine group. Metal chelates that can be detected by electrochemiluminescense are also preferred signal-emitting groups, with particular preference being given to ruthenium chelates, e.g. a ruthenium (bispyridyl)₃²⁺ chelate. Suitable ruthenium labeling groups are described, for example, in EP 0 580 979, WO 90/05301, WO 90/11511, and WO 92/14138. For direct detection the labeling group can be selected from any known detectable marker groups, such as dyes, luminescent labeling groups such as chemiluminescent groups, e.g. acridinium esters or dioxetanes, or fluorescent dyes, e.g. fluorescein, coumarin, rhodamine, oxazine, resorufin, cyanine and derivatives thereof. Other examples of labeling groups are luminescent metal complexes, such as ruthenium or europium complexes, enzymes, e.g. as used for ELISA or for CEDIA (Cloned Enzyme Donor Immunoassay, e.g. EP-A-0 061 888), and radioisotopes.

Indirect detection systems comprise, for example, that the detection reagent, e.g., the detection antibody is labeled with a first partner of a binding pair. Examples of suitable binding pairs are antigen/antibody, biotin or biotin analogues such as aminobiotin, iminobiotin or desthiobiotin/avidin or Streptavidin, sugar/lectin, nucleic acid or nucleic acid analogue/complementary nucleic acid, and receptor/ligand, e.g., steroid hormone receptor/steroid hormone. In one preferred embodiment, the first binding pair members comprise hapten, antigen and hormone. In one preferred embodiment, the hapten is selected from the group consisting of digoxin, digoxygenin and biotin and analogues thereof. The second partner of such binding pair, e.g. an antibody, Streptavidin, etc., usually is labeled to allow for direct detection, e.g., by the labels as mentioned above.

The term “immunoassay” denotes any technique that utilizes specifically binding molecules, such as antibodies, to capture and/or detect a specific target for qualitative or quantitative analysis. In general, an immunoassay is characterized by the following steps: 1) immobilization or capture of the analyte and 2) detection and measuring the analyte. The analyte can be captured, i.e. bound, on any solid surface, such as e.g. a membrane, plastic plate, or some other solid surface.

Immunoassays can be performed generally in three different formats. One is with direct detection, one with indirect detection, or by a sandwich assay. The direct detection immunoassay uses a detection (or tracer) antibody that can be measured directly. An enzyme or other molecule allows for the generation of a signal that will produce a color, fluorescence, or luminescence that allow the signal to be visualized or measured (radioisotopes can also be used, although it is not commonly used today). In an indirect assay a primary antibody that binds to the analyte is used to provide a defined target for a secondary antibody (tracer antibody) that specifically binds to the target provided by the primary antibody (referred to as detector or tracer antibody). The secondary antibody generates the measurable signal. The sandwich assay makes use of two antibodies, a capture and a trace (detector) antibody. The capture antibody is used to bind (immobilize) analyte from solution or bind to it in solution. This allows the analyte to be specifically removed from the sample. The tracer (detector) antibody is used in a second step to generate a signal (either directly or indirectly as described above). The sandwich format requires two antibodies each with a distinct epitope on the target molecule. In addition, they must not interfere with one another as both antibodies must be bound to the target at the same time.

The term “free antigen” denotes the antigen that can be specifically bound by a binding specificity of an antibody but which is currently not bound to this binding specificity. In certain embodiments of the invention, the free antigen is a not-antibody bound antigen or a non-antibody complexed antigen, i.e. an antigen that is not in a covalent or non-covalent complex with a (any) therapeutic antibody.

The principles of different immunoassays are described, for example, by Hage, D. S. (Anal. Chem. 71 (1999) 294R-304R). Lu, B., et al. (Analyst 121 (1996) 29R-32R) report the orientated immobilization of antibodies for the use in immunoassays. Avidin-biotin-mediated immunoassays are reported, for example, by Wilchek, M., and Bayer, E. A., in Methods Enzymol. 184 (1990) 467-469.

The term “biparatopic antibody” denotes an antibody having at least two binding sites and specifically binding to two, non-overlapping epitopes on the same antigen.

### Embodiments of the Method According to the Invention

A single interaction between a first binding site of a therapeutic antibody and the antigen results in the formation of an antigen-antibody-complex. The half-life of this single interaction depends on a simple affinity driven interaction, i.e. without avid participation. Only by the interaction of the second binding site of the antibody with the antigen a long-time stable complex with affine and avid binding interactions is formed.

By this characteristic the determination of free antigen in the presence of the therapeutic antibody is not straight forward.

Generally, a bridging principle is used for the determination of free antigen in a sample, e.g. obtained from an experimental animal of human. Thereby the antigen is bound to (captured on) a solid phase (via a first epitope) by the use of a so-called capture antibody and detected via a second non-overlapping epitope by the use of a so-called tracer antibody.

Thus, a positive assay result can only be obtained if the bridged complex, which comprises two exclusively affinity-driven interactions, is sufficiently stable.

Additionally, if the assay is required to detect free antigen in the presence of the therapeutic antibody (or even more complex in the presence of different therapeutic antibodies binding to the same antigen), the capture and the tracer antibody need to bind to the same or to at least partly overlapping epitopes as the therapeutic antibody does/the therapeutic antibodies do.

Additionally, the formation of the detection complex shall not change the fraction of the free antigen, i.e., e.g., by replacing the therapeutic antibody. Without being bound by this theory, it is assumed that the capture or detection antibody should not influence the amount of the detection complex. It is assumed that the incubation time has to be aligned with the off-rate of the complex; preferably it has to be shorter.

Thus, the assay shall allow for sensitive determination of free antigen of a therapeutic antibody even in the case of short half-lives of the individual interactions and the presence of the therapeutic antibody.

The invention is based at least in part on the finding that for the determination of the free antigen of a therapeutic antibody an assay with short interaction times without sample dilution achieves the best results.

The following exemplification of the method according to the invention is presented using an exemplary bispecific anti-CCL2 antibody. This is presented as mere exemplification and shall not be construed as limitation of the method according to the current invention. The true scope is set forth in the appended claims.

The corresponding examples are Examples 4 to 10. These show the properties of an immunoassay for the determination of free CCL2 (not in a complex with an anti-CCL2 antibody) with a sensitivity of 10 pg/mL to support a proof of concept (POC) study in cynomolgus monkey.

It has been found that
in ELISA format the combination of dilution and long incubation times results in false results (a value for the ratio of 1 is to be expected if no effect of dilution exists; see Example 4):

| | ratio of determined CCL2 value at dilution 1 to 40 | | | | | | | | |
|---|---|---|---|---|---|---|---|---|---|
| | to determined CCL2 value at dilution 1 to 4 | | | | | | | | |
| ng/ml | ng/mL bispecific anti-CCL2 antibody (↓) | | | | | | | | |
| cyCCL2 (↓) | 50000 | 12500 | 3125 | 781 | 195 | 49 | 12 | 3 | 0 |
| 500.00 | 10.9 | 8.2 | | | | | | | |
| 100.00 | 12.5 | 8.8 | 6.6 | | | | | | |
| 20.00 | | 8.4 | 6.2 | 5.2 | | | | | |
| 4.00 | | | 6.3 | 5.0 | 3.0 | | | | |
| 0.80 | | | | | 3.1 | 1.8 | 1.3 | 1.1 | 1.1 |
| 0.16 | | | | | | | | | |
| 0.032 | | | | | | | | | |
| 0 | | | | | | | | | |

in ELISA format a short incubation time of about 3-4 min. without dilution (100% horse serum) results in an assay working range of from 20 pg/mL antigen to 1000 pg/mL antigen (see FIG. 1 and Example 5):

| signal | | | | | |
|---|---|---|---|---|---|
| [AU] | | | | | |
| ng/ml | incubation time | | | | |
| cyCCL2 | 12 min. | 9 min. | 6 min. | 3.5 min. | 1.25 min |
| 1000.00 | 3.01 | 2.86 | 2.65 | 2.31 | 1.81 |
| 500.00 | 2.37 | 2.01 | 1.48 | 1.25 | 1.02 |
| 250.00 | 1.15 | 1.03 | 0.89 | 0.72 | 0.52 |
| 125.00 | 0.72 | 0.61 | 0.54 | 0.44 | 0.29 |
| 62.500 | 0.38 | 0.32 | 0.28 | 0.22 | 0.16 |
| 31.250 | 0.21 | 0.19 | 0.17 | 0.16 | 0.10 |
| 15.625 | 0.14 | 0.12 | 0.10 | 0.10 | 0.07 |
| 7.813 | 0.10 | 0.08 | 0.08 | 0.07 | 0.07 |
| 3.906 | 0.07 | 0.07 | 0.06 | 0.05 | 0.06 |
| 0.000 | 0.05 | 0.05 | 0.05 | 0.05 | 0.05 |

in a nanoliter-scale, microfluidic, affinity flow-through format with laser-induced fluorescence detection format despite a manufacturer's minimal required dilution of 1:2 the assay works without dilution and has a working range of 313 pg/mL to 40 000 pg/mL and in a nanoliter-scale, microfluidic, affinity flow-through format with laser-induced fluorescence detection format despite a manufacturer's minimal required dilution of 1:2 the assay works without dilution and has a LLOQ of 10 pg/mL if the detection antibody is directly labelled with the dye (see FIG. 2 and Example 7).

| | capture antibody: | | | |
|---|---|---|---|---|
| | anti-CCL2 antibody-Bi | | anti-CCL2 antibody-Bi | |
| | detection antibody: | | | |
| | | | pre-incubated humanized | |
| | | | 11K2-Dig + | |
| | humanized 11K2- | | mAb<Dig>M-1.71.256- | |
| cyCCL2 c | Alexa 647 | | IgG-Alexa 647 | |
| [pg/mL] | average | cv | average | cv |
| 51200 | 480.73 | 4.3% | 266.68 | 0.8% |
| 12800 | 200.33 | 0.5% | 86.47 | 3.3% |
| 3200 | 62.07 | 1.1% | 27.75 | 2.9% |
| 800 | 18.02 | 3.4% | 7.86 | 6.9% |
| 200 | 4.92 | 6.5% | 2.38 | 0.7% |
| 50 | 1.27 | 1.6% | 0.88 | 4.3% |
| 12.5 | 0.34 | 5.2% | 0.50 | 3.9% |
| 0 | 0.09 | 27.0% | 0.39 | 6.3% |

by using capture and detection antibodies with a non-human backbone a) false positive and b) false negative results are avoided
   as ADAs (anti-drug antibodies) directed against constant regions in human IgG might be able to build a bridge between human capture and detection antibody resulting in the induction of false positive free antigen assay results
   ADAs directed against CDRs of drug antibody might be able to bind to the capture antibody in a neutralizing manner causing false negative free antigen assay results as the capture antibody is not able to capture free antigen anymore,
   the assay shows the same results with the non-human backbone antibodies that are different from the humanized ones but still bind to the same epitope as shown in the following table:

| | | CCL2 recovery; free [%] | | | |
|---|---|---|---|---|---|
| pg/mL | ng/ml | CNTO0888- | CKLO2- | CKLO2- | CKLO2- |
| CCL2 | drug | SG1 | SG1 | SG1100 | SG1095 |

capture antibody: CNTO0888-Bi; detection antibody: 11K2-Alexa 647

| | | | | | |
|---|---|---|---|---|---|
| 5000 | 500 | 1.2% | 0.4% | 0.3% | 0.3% |
| 5000 | 250 | 2.1% | 0.7% | 0.5% | 0.6% |
| 5000 | 125 | 4.4% | 2.5% | 1.9% | 2.0% |
| 5000 | 62.5 | 13.3% | 36.1% | 28.8% | 34.3% |
| 5000 | 0 | 96.7% | 104.9% | 99.4% | 106.3% |

capture antibody: 2F6-Bi; detection antibody: 1H11-Alexa 647

| | | | | | |
|---|---|---|---|---|---|
| 5000 | 500 | 1.1% | 0.3% | 0.3% | 0.3% |
| 5000 | 250 | 2.1% | 0.7% | 0.5% | 0.6% |
| 5000 | 125 | 4.7% | 2.5% | 2.0% | 1.9% |
| 5000 | 62.5 | 12.7% | 36.1% | 27.9% | 33.3% |
| 5000 | 0 | 92.7% | 97.9% | 97.7% | 100.9% |

The assay according to the invention has been used to analyze samples from a cynomolgus pharmacokinetic study. The results obtained for the control samples are presented in the following table (see also Example 8):

| | Measured CCL2 [pg/mL] | | | | |
|---|---|---|---|---|---|
| | | CPS with CKL02-SG1095 | | CPS with CNTO0888 | |
| Run | CPS | 10 μg/mL | 15 ng/ml | 10 μg/mL | 7.5 ng/mL |
| 1 | 586.7 | 13.5 | 128.8 | | |
| 2 | 597.3 | 13.3 | 134.1 | | |
| 3 | 560.5 | 12.0 | 144.0 | | |
| 4 | 570.5 | 14.8 | 116.7 | | |
| 5 | 600.5 | 13.4 | 130.6 | | |
| 6 | 586.2 | | | 15.1 | 272.6 |
| 7 | 593.3 | | | 13.7 | 264.0 |
| 8 | 563.0 | | | 14.8 | 242.7 |
| 9 | 657.4 | 14.4 | 139.4 | | |
| 10 | 568.6 | 17.1 | 142.0 | | |
| 11 | 573.0 | 12.2 | 122.1 | | |
| 12 | 552.5 | 14.6 | 135.1 | | |
| av | 584 | 14 | 133 | 15 | 260 |
| cv | 5% | 11% | 7% | 5% | 6% |

The same assay setup has been used for the determination of human antigen in B16 mice (see FIG. 4 and Example 9).
In certain embodiments of the invention, the antigen is human CCL2 and the antibody is a bispecific anti-CCL2 antibody binding to two different epitopes on human CCL2.
In certain embodiments of the invention, the bispecific antibody comprises a first antigen-binding site that (specifically) binds to a first epitope on human CCL2 and a second different antigen-binding site that (specifically) binds a second epitope on human CCL2.
In certain embodiments of the invention, the bispecific antibody comprises a first antigen-binding site that (specifically) binds to a first epitope on human CCL2 and a second different antigen-binding site that (specifically) binds a second epitope on human CCL2,
   wherein
   A) i) said first antigen-binding site comprises
      a VH domain comprising (a) a CDR-H1 comprising the amino acid sequence of SEQ ID NO: 142, (b) a CDR-H2 comprising the amino acid sequence of SEQ ID NO: 143, and (c) a CDR-H3 comprising the amino acid sequence of SEQ ID NO: 144;
      and a VL domain comprising (d) a CDR-L1 comprising the amino acid sequence of SEQ ID NO: 145; (e) a CDR-L2 comprising the amino acid sequence of SEQ ID NO: 146, and (f) a CDR-L3 comprising the amino acid sequence of SEQ ID NO: 147; and
   ii) said second antigen-binding site comprises
      a VH domain comprising (a) a CDR-H1 comprising the amino acid sequence of SEQ ID NO: 150, (b) a CDR-H2 comprising the amino acid sequence of SEQ ID NO: 151, and (c) a CDR-H3 comprising the amino acid sequence of SEQ ID NO: 152;
      and a VL domain comprising (d) a CDR-L1 comprising the amino acid sequence of SEQ ID NO: 153; (e) a CDR-L2 comprising the amino acid sequence of SEQ ID NO: 154, and (f) a CDR-L3 comprising the amino acid sequence of SEQ ID NO: 155;
   or
   B) i) said first antigen-binding site comprises
      a VH domain comprising (a) a CDR-H1 comprising the amino acid sequence of SEQ ID NO: 142, (b) a CDR-H2 comprising the amino acid sequence of SEQ ID NO: 143, and (c) a CDR-H3 comprising the amino acid sequence of SEQ ID NO: 144;
      and a VL domain comprising (d) a CDR-L1 comprising the amino acid sequence of SEQ ID NO: 145; (e) a CDR-L2 comprising the amino acid sequence of SEQ ID NO: 146, and (f) a CDR-L3 comprising the amino acid sequence of SEQ ID NO: 147; and
   ii) said second antigen-binding site comprises
      a VH domain comprising (a) a CDR-H1 comprising the amino acid sequence of SEQ ID NO: 130, (b) a CDR-H2 comprising the amino acid sequence of SEQ ID NO: 131, and (c) a CDR-H3 comprising the amino acid sequence of SEQ ID NO: 132;
      and a VL domain comprising (d) a CDR-L1 comprising the amino acid sequence of SEQ ID NO: 133; (e) a CDR-L2 comprising the amino acid sequence of SEQ ID NO: 134, and (f) a CDR-L3 comprising the amino acid sequence of SEQ ID NO: 135;
   C) i) said first antigen-binding site comprises
      a VH domain comprising (a) a CDR-H1 comprising the amino acid sequence of SEQ ID NO: 142, (b) a CDR-H2 comprising the amino acid sequence of SEQ ID NO: 143, and (c) a CDR-H3 comprising the amino acid sequence of SEQ ID NO: 144;
      and a VL domain comprising (d) a CDR-L1 comprising the amino acid sequence of SEQ ID NO: 145; (e) a CDR-L2 comprising the amino acid sequence of SEQ ID NO: 146, and (f) a CDR-L3 comprising the amino acid sequence of SEQ ID NO: 147; and
      ii) said second antigen-binding site comprises
      a VH domain comprising (a) a CDR-H1 comprising the amino acid sequence of SEQ ID NO: 124, (b) a CDR-H2 comprising the amino acid sequence of SEQ ID NO: 125, and (c) a CDR-H3 comprising the amino acid sequence of SEQ ID NO: 126;
      and a VL domain comprising (d) a CDR-L1 comprising the amino acid sequence of SEQ ID NO: 127; (e) a CDR-L2 comprising the amino acid sequence of SEQ ID NO: 128, and (f) a CDR-L3 comprising the amino acid sequence of SEQ ID NO: 129;
   or
   D) i) said first antigen-binding site comprises
      a VH domain comprising (a) a CDR-H1 comprising the amino acid sequence of SEQ ID NO: 130, (b) a CDR-H2 comprising the amino acid sequence of SEQ ID NO: 131, and (c) a CDR-H3 comprising the amino acid sequence of SEQ ID NO: 132;
      and a VL domain comprising (d) a CDR-L1 comprising the amino acid sequence of SEQ ID NO: 133; (e) a CDR-L2 comprising the amino acid sequence of SEQ ID NO: 134, and (f) a CDR-L3 comprising the amino acid sequence of SEQ ID NO: 135; and
      ii) said second antigen-binding site comprises
      a VH domain comprising (a) a CDR-H1 comprising the amino acid sequence of SEQ ID NO: 150, (b) a CDR-H2 comprising the amino acid sequence of SEQ ID NO: 151, and (c) a CDR-H3 comprising the amino acid sequence of SEQ ID NO: 152;
      and a VL domain comprising (d) a CDR-L1 comprising the amino acid sequence of SEQ ID NO: 153; (e) a CDR-L2 comprising the amino acid sequence of SEQ ID NO: 154, and (f) a CDR-L3 comprising the amino acid sequence of SEQ ID NO: 155;
   or
   E) i) said first antigen-binding site comprises
      a VH domain comprising (a) a CDR-H1 comprising the amino acid sequence of SEQ ID NO: 136, (b) a CDR-H2 comprising the amino acid sequence of SEQ ID NO: 137, and (c) a CDR-H3 comprising the amino acid sequence of SEQ ID NO: 138;
      and a VL domain comprising (d) a CDR-L1 comprising the amino acid sequence of SEQ ID NO: 139; (e) a CDR-L2 comprising the amino acid sequence of SEQ ID NO: 140, and (f) a CDR-L3 comprising the amino acid sequence of SEQ ID NO: 141; and
      ii) said second antigen-binding site comprises
      a VH domain comprising (a) a CDR-H1 comprising the amino acid sequence of SEQ ID NO: 150, (b) a CDR-H2 comprising the amino acid sequence of SEQ ID NO: 151, and (c) a CDR-H3 comprising the amino acid sequence of SEQ ID NO: 152; and
      a VL domain comprising (d) a CDR-L1 comprising the amino acid sequence of SEQ ID NO: 153; (e) a CDR-L2 comprising the amino acid sequence of SEQ ID NO: 154, and (f) a CDR-L3 comprising the amino acid sequence of SEQ ID NO: 155;
   or
   F) i) said first antigen-binding site comprises
      a VH domain comprising (a) a CDR-H1 comprising the amino acid sequence of SEQ ID NO: 158, (b) a CDR-H2 comprising the amino acid sequence of SEQ ID NO: 159, and (c) a CDR-H3 comprising the amino acid sequence of SEQ ID NO: 160;
      and a VL domain comprising (d) a CDR-L1 comprising the amino acid sequence of SEQ ID NO: 161; (e) a CDR-L2 comprising the amino acid sequence of SEQ ID NO: 162, and (f) a CDR-L3 comprising the amino acid sequence of SEQ ID NO: 163; and
      ii) said second antigen-binding site comprises
      a VH domain comprising (a) a CDR-H1 comprising the amino acid sequence of SEQ ID NO: 150, (b) a CDR-H2 comprising the amino acid sequence of SEQ ID NO: 151, and (c) a CDR-H3 comprising the amino acid sequence of SEQ ID NO: 152; and
      a VL domain comprising (d) a CDR-L1 comprising the amino acid sequence of SEQ ID NO: 153; (e) a CDR-L2 comprising the amino acid sequence of SEQ ID NO: 154, and (f) a CDR-L3 comprising the amino acid sequence of SEQ ID NO: 155;
   or
   G) i) said first antigen-binding site comprises
      a VH domain comprising (a) a CDR-H1 comprising the amino acid sequence of SEQ ID NO: 124, (b) a CDR-H2 comprising the amino acid sequence of SEQ ID NO: 125, and (c) a CDR-H3 comprising the amino acid sequence of SEQ ID NO: 126;
      and a VL domain comprising (d) a CDR-L1 comprising the amino acid sequence of SEQ ID NO: 127; (e) a CDR-L2 comprising the amino acid sequence of SEQ ID NO: 128, and (f) a CDR-L3 comprising the amino acid sequence of SEQ ID NO: 129; and
      ii) said second antigen-binding site comprises
      a VH domain comprising (a) a CDR-H1 comprising the amino acid sequence of SEQ ID NO: 130, (b) a CDR-H2 comprising the amino acid sequence of SEQ ID NO: 131, and (c) a CDR-H3 comprising the amino acid sequence of SEQ ID NO: 132;
      and a VL domain comprising a (d) CDR-L1 comprising the amino acid sequence of SEQ ID NO: 133; (e) a CDR-L2 comprising the amino acid sequence of SEQ ID NO: 134, and (f) a CDR-L3 comprising the amino acid sequence of SEQ ID NO: 135;
   or
   H) i) said first antigen-binding site comprises
      a VH domain comprising (a) a CDR-H1 comprising the amino acid sequence of SEQ ID NO: 124, (b) a CDR-H2 comprising the amino acid sequence of SEQ ID NO: 125, and (c) a CDR-H3 comprising the amino acid sequence of SEQ ID NO: 126;
      and a VL domain comprising (d) a CDR-L1 comprising the amino acid sequence of SEQ ID NO: 127; (e) a CDR-L2 comprising the amino acid sequence of SEQ ID NO: 128, and (f) a CDR-L3 comprising the amino acid sequence of SEQ ID NO: 129; and
      ii) said second antigen-binding site comprises
      a VH domain comprising (a) a CDR-H1 comprising the amino acid sequence of SEQ ID NO: 136, (b) a CDR-H2 comprising the amino acid sequence of SEQ ID NO: 137, and (c) a CDR-H3 comprising the amino acid sequence of SEQ ID NO: 138;
      and a VL domain comprising (d) a CDR-L1 comprising the amino acid sequence of SEQ ID NO: 139; (e) a CDR-L2 comprising the amino acid sequence of SEQ ID NO: 140, and (f) a CDR-L3 comprising the amino acid sequence of SEQ ID NO: 141;
   or
   I) i) said first antigen-binding site comprises
      a VH domain comprising (a) a CDR-H1 comprising the amino acid sequence of SEQ ID NO: 118, (b) a CDR-H2 comprising the amino acid sequence of SEQ ID NO: 119, and (c) a CDR-H3 comprising the amino acid sequence of SEQ ID NO: 120;
      and a VL domain comprising (d) a CDR-L1 comprising the amino acid sequence of SEQ ID NO: 121; (e) a CDR-L2 comprising the amino acid sequence of SEQ ID NO: 122, and (f) a CDR-L3 comprising the amino acid sequence of SEQ ID NO: 123; and
      ii) said second antigen-binding site comprises
      a VH domain comprising (a) a CDR-H1 comprising the amino acid sequence of SEQ ID NO: 136, (b) a CDR-H2 comprising the amino acid sequence of SEQ ID NO: 137, and (c) a CDR-H3 comprising the amino acid sequence of SEQ ID NO: 138;
      and a VL domain comprising (d) a CDR-L1 comprising the amino acid sequence of SEQ ID NO: 139; (e) a CDR-L2 comprising the amino acid sequence of SEQ ID NO: 140, and (f) a CDR-L3 comprising the amino acid sequence of SEQ ID NO: 141.
In certain embodiments of the invention, the bispecific antibody comprises an Fc domain of human IgG1 isotype.
In certain embodiments of the invention, the bispecific antibody comprises constant heavy chain domain of human IgG1 isotype.
In certain embodiments of the invention, the bispecific antibody is an (isolated) bispecific antibody comprising a first antigen-binding site that (specifically) binds to a first epitope on human CCL2 and a second antigen-binding site that (specifically) binds a second epitope on human CCL2, wherein i) said first antigen-binding site binds to same epitope on CCL2 as an antibody comprising a VH domain comprising the amino acid sequence of SEQ ID NO: 148, wherein the VH domain comprises (a) a CDR-H1 comprising the amino acid sequence of SEQ ID NO: 142, (b) a CDR-H2 comprising the amino acid sequence of SEQ ID NO: 143, and (c) a CDR-H3 comprising the amino acid sequence of SEQ ID NO: 144;

and a VL domain comprising the amino acid sequence of SEQ ID NO: 149, wherein the VL domain comprises (d) a CDR-L1 comprising the amino acid sequence of SEQ ID NO: 145; (e) a CDR-L2 comprising the amino acid sequence of SEQ ID NO: 146, and (0 a CDR-L3 comprising the amino acid sequence of SEQ ID NO: 147; and ii) said second antigen-binding site binds to same epitope on CCL2 as an antibody comprising a VH domain comprising the amino acid sequence of SEQ ID NO: 156, wherein the VH domain comprises (a) a CDR-H1 comprising the amino acid sequence of SEQ ID NO: 150, (b) a CDR-H2 comprising the amino acid sequence of SEQ ID NO: 151, and (c) a CDR-H3 comprising the amino acid sequence of SEQ ID NO: 152; and a VL domain comprising the amino acid sequence of SEQ ID NO: 157, wherein the VL domain comprises (d) a CDR-L1 comprising the amino acid sequence of SEQ ID NO: 153; (e) a CDR-L2 comprising the amino acid sequence of SEQ ID NO: 154, and (0 a CDR-L3 comprising the amino acid sequence of SEQ ID NO: 155.
In certain embodiments of the invention, the bispecific antibody is an (isolated) bispecific antibody comprising a first antigen-binding site that (specifically) binds to a first epitope on human CCL2 and a second antigen-binding site that (specifically) binds a second epitope on human CCL2,
   wherein
   i) said first antigen-binding site comprises
      a VH domain comprising (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and
      a VL domain comprising (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169;
      and
   ii) said second antigen-binding site comprises
      a VH domain comprising (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein Xis D or E;
      and
      a VL domain comprising (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R.
In certain embodiments of the invention, the bispecific antibody is an (isolated) bispecific antibody comprising a first antigen-binding site that (specifically) binds to a first epitope on human CCL2 and a second antigen-binding site that (specifically) binds a second epitope on human CCL2,
   wherein
   i) said first antigen-binding site comprises
      a VH domain comprising (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, (c) a CDR-H3 comprising the amino acid sequence YDAHYGELDF of SEQ ID NO: 166, (d) a FR-H1 comprising the amino acid sequence QVQLVQSGAEVKKPGSSVKVSCKASGGTF of SEQ ID NO: 170, (e) a FR-H2 comprising the amino acid sequence WVRQAPGQGLEWMG of SEQ ID NO: 171, (f) a FR-H3 comprising the amino acid sequence RVTITADESTSTAYMELSSLRSEDTAVY YCAR of SEQ ID NO: 172, and (g) a FR-H4 comprising the amino acid sequence WGQGTLVTVSS of SEQ ID NO: 173;
      and
      a VL domain comprising (h) a CDR-L1 comprising the amino acid sequence RASQHVSDAYLA of SEQ ID NO: 167; (i) a CDR-L2 comprising the amino acid sequence DASDRAE of SEQ ID NO: 168, and (j) a CDR-L3 comprising the amino acid sequence HQYIHLHSFT of SEQ ID NO: 169, (k) a FR-L1 comprising the amino acid sequence EIVLTQSPATLSLSPGERATLSC of SEQ ID NO: 174, (l) a FR-L2 comprising the amino acid sequence WYQQKPGQAPRLLIY of SEQ ID NO: 175, (m) a FR-L3 comprising the amino acid sequence GVPARFSGSGSGTDFTLTISSLEPEDFAVYYC of SEQ ID NO: 176, and (n) a FR-L4 comprising the amino acid sequence GQGTKVEIK of SEQ ID NO: 177;
      and
   ii) said second antigen-binding site comprises
      a VH domain comprising (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, (c) a CDR-H3 comprising the amino acid sequences GVFGFFXH of SEQ ID NO: 185, wherein Xis D or E, (d) a FR-H1 comprising the amino acid sequence QVQLVQSGAEVKKPGSSVKVSCKASGLTIS of SEQ ID NO: 189, (e) a FR-H2 comprising the amino acid sequence WVRQAPGQGLEWMG of SEQ ID NO: 190, (f) a FR-H3 comprising the amino acid sequence RVTITADTSTSTAYMELSSLRSEDTAVYYCAR of SEQ ID NO: 191, and (g) a FR-H4 comprising the amino acid sequence WGQGTTVTVSS of SEQ ID NO: 192;
      and
      a VL domain comprising (h) a CDR-L1 comprising the amino acid sequence KAX¹EDIYNRX²A of SEQ ID NO: 186, wherein X¹ is F or T and X² is R or L, (i) a CDR-L2 comprising the amino acid sequence GATSLEH of SEQ ID NO: 187, (j) a CDR-L3 comprising the amino acid sequence QQFXSAPYT of SEQ ID NO: 188, wherein X is W or R, (k) a FR-L1 comprising the amino acid sequence DIQMTQSPSSLSASVGDRVTITC of SEQ ID NO: 193, (1) a FR-L2 comprising the amino acid sequence WYQQKPGKAPKLLIH of SEQ ID NO: 194, (m) a FR-L3 comprising the amino acid sequence GVPSRFSGSGSGTDYTLTISSLQPEDFATYYC of SEQ ID NO: 195, and (n) a FR-L4 comprising the amino acid sequence FGGGTKVEIK of SEQ ID NO: 196.
In certain embodiments of the invention, the bispecific antibody is an (isolated) bispecific antibody comprising a first antigen-binding site that (specifically) binds to a first epitope on human CCL2 and a second antigen-binding site that (specifically) binds a second epitope on human CCL2,
   wherein
   A) i) said first antigen-binding site comprises
      a VH domain comprising the amino acid sequence of SEQ ID NO: 178; and a VL domain comprising the amino acid sequence of SEQ ID NO: 182; and
      ii) said second antigen-binding site comprises a VH domain comprising the amino acid sequence of SEQ ID NO: 197; and a VL domain comprising the amino acid sequence of SEQ ID NO: 200;
   or
   B) i) said first antigen-binding site comprises
      a VH domain comprising the amino acid sequence of SEQ ID NO: 178; and a VL domain comprising the amino acid sequence of SEQ ID NO: 182; and
      ii) said second antigen-binding site comprises
      a VH domain comprising the amino acid sequence of SEQ ID NO: 198; and a VL domain comprising the amino acid sequence of SEQ ID NO: 200;
   or
   C) i) said first antigen-binding site comprises
      a VH domain comprising the amino acid sequence of SEQ ID NO: 178; and a VL domain comprising the amino acid sequence of SEQ ID NO: 182; and
      ii) said second antigen-binding site comprises
      a VH domain comprising the amino acid sequence of SEQ ID NO: 197; and a VL domain comprising the amino acid sequence of SEQ ID NO: 201;
   or
   D) i) said first antigen-binding site comprises
      a VH domain comprising the amino acid sequence of SEQ ID NO: 179; and a VL domain comprising the amino acid sequence of SEQ ID NO: 182; and
      ii) said second antigen-binding site comprises
      a VH domain comprising the amino acid sequence of SEQ ID NO: 197; and a VL domain comprising the amino acid sequence of SEQ ID NO: 201;
   or
   E) i) said first antigen-binding site comprises a VH domain comprising the amino acid sequence of SEQ ID NO: 180; and a VL domain comprising the amino acid sequence of SEQ ID NO: 182; and
      ii) said second antigen-binding site comprises a VH domain comprising the amino acid sequence of SEQ ID NO: 197; and a VL domain comprising the amino acid sequence of SEQ ID NO: 200;
   or
   F) i) said first antigen-binding site comprises
      a VH domain comprising the amino acid sequence of SEQ ID NO: 180; and a VL domain comprising the amino acid sequence of SEQ ID NO: 182; and
      ii) said second antigen-binding site comprises
      a VH domain comprising the amino acid sequence of SEQ ID NO: 197; and a VL domain comprising the amino acid sequence of SEQ ID NO: 201;
   or
   G) i) said first antigen-binding site comprises
      a VH domain comprising the amino acid sequence of SEQ ID NO: 180; and a VL domain comprising the amino acid sequence of SEQ ID NO: 182; and
      ii) said second antigen-binding site comprises
      a VH domain comprising the amino acid sequence of SEQ ID NO: 199; and a VL domain comprising the amino acid sequence of SEQ ID NO: 200;
   H) i) said first antigen-binding site comprises
      a VH domain comprising the amino acid sequence of SEQ ID NO: 180; and a VL domain comprising the amino acid sequence of SEQ ID NO: 182; and
      ii) said second antigen-binding site comprises
      a VH domain comprising the amino acid sequence of SEQ ID NO: 198; and a VL domain comprising the amino acid sequence of SEQ ID NO: 200;
   or
   I) i) said first antigen-binding site comprises
      a VH domain comprising the amino acid sequence of SEQ ID NO: 179; and a VL domain comprising the amino acid sequence of SEQ ID NO: 182; and
      ii) said second antigen-binding site comprises
      a VH domain comprising the amino acid sequence of SEQ ID NO: 197; and a VL domain comprising the amino acid sequence of SEQ ID NO: 200;
   or
   J) i) said first antigen-binding site comprises
      a VH domain comprising the amino acid sequence of SEQ ID NO: 179; and a VL domain comprising the amino acid sequence of SEQ ID NO: 182; and
      ii) said second antigen-binding site comprises
      a VH domain comprising the amino acid sequence of SEQ ID NO: 199; and a VL domain comprising the amino acid sequence of SEQ ID NO: 200;
   or
   K) i) said first antigen-binding site comprises
      a VH domain comprising the amino acid sequence of SEQ ID NO: 179; and a VL domain comprising the amino acid sequence of SEQ ID NO: 182; and
      ii) said second antigen-binding site comprises
      a VH domain comprising the amino acid sequence of SEQ ID NO: 198; and a VL domain comprising the amino acid sequence of SEQ ID NO: 200;
   or
   L) i) said first antigen-binding site comprises
      a VH domain comprising the amino acid sequence of SEQ ID NO: 181; and a VL domain comprising the amino acid sequence of SEQ ID NO: 182; and
      ii) said second antigen-binding site comprises
      a VH domain comprising the amino acid sequence of SEQ ID NO: 197; and a VL domain comprising the amino acid sequence of SEQ ID NO: 200;
   or
   M) i) said first antigen-binding site comprises
      a VH domain comprising the amino acid sequence of SEQ ID NO: 181; and a VL domain comprising the amino acid sequence of SEQ ID NO: 182; and
      ii) said second antigen-binding site comprises
      a VH domain comprising the amino acid sequence of SEQ ID NO: 197; and a VL domain comprising the amino acid sequence of SEQ ID NO: 201;
   or
   N) i) said first antigen-binding site comprises
      a VH domain comprising the amino acid sequence of SEQ ID NO: 181; and a VL domain comprising the amino acid sequence of SEQ ID NO: 182; and
      ii) said second antigen-binding site comprises
      a VH domain comprising the amino acid sequence of SEQ ID NO: 199; and a VL domain comprising the amino acid sequence of SEQ ID NO: 200;
   or
   O) i) said first antigen-binding site comprises
      a VH domain comprising the amino acid sequence of SEQ ID NO: 181; and a VL domain comprising the amino acid sequence of SEQ ID NO: 182; and
      ii) said second antigen-binding site comprises
      a VH domain comprising the amino acid sequence of SEQ ID NO: 198; and a VL domain comprising the amino acid sequence of SEQ ID NO: 200;
   or
   P) i) said first antigen-binding site comprises
      a VH domain comprising the amino acid sequence of SEQ ID NO: 178; and a VL domain comprising the amino acid sequence of SEQ ID NO: 182; and
      ii) said second antigen-binding site comprises
      a VH domain comprising the amino acid sequence of SEQ ID NO: 199; and a VL domain comprising the amino acid sequence of SEQ ID NO: 200.
In certain embodiments of the invention, the bispecific antibody is an (isolated) bispecific antibody comprising a first antigen-binding site that (specifically) binds to a first epitope on human CCL2 and a second antigen-binding site that (specifically) binds a second epitope on human CCL2,
   wherein
   A) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 178, wherein the VH domain comprises (a) a CDR-H1 comprising the amino acid sequence SHYGXS of SEQ ID NO: 164, wherein X is I, (b) a CDR-H2 comprising the amino acid sequence GX¹IX²IFX³TANYAQKFQG of SEQ ID NO: 165, wherein X¹ is V, X² is P, and X³ is H, and (c) a CDR-H3 comprising the amino acid sequence YDAHYGELDF of SEQ ID NO: 166; and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D, and (c) a CDR-H3 comprising the amino acid sequences GVFGFFXH of SEQ ID NO: 185, wherein X is D;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F and X² is R, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W;
   or
   B) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 178, wherein the VH domain comprises (a) a CDR-H1 comprising the amino acid sequence SHYGXS of SEQ ID NO: 164, wherein X is I, (b) a CDR-H2 comprising the amino acid sequence GX¹IX²IFX³TANYAQKFQG of SEQ ID NO: 165, wherein X¹ is V, X² is P, and X³ is H, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 198, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D, and (c) a CDR-H3 comprising the amino acid sequences GVFGFFXH of SEQ ID NO: 185, wherein X is E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F and X² is R, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W;
   or
   C) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 178, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein Xis D or E;
      and a VL domain sequence having at least 90%, 91%, 92%, 93%, 94%, 95%, 96%, 97%, 98%, 99%, or 100% sequence identity to the amino acid sequence of SEQ ID NO: 201, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   D) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 179, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein Xis D or E;
      and a VL domain sequence having at least 90%, 91%, 92%, 93%, 94%, 95%, 96%, 97%, 98%, 99%, or 100% sequence identity to the amino acid sequence of SEQ ID NO: 201, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   E) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 180, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   F) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 180, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein Xis D or E;
      and a VL domain sequence having at least 90%, 91%, 92%, 93%, 94%, 95%, 96%, 97%, 98%, 99%, or 100% sequence identity to the amino acid sequence of SEQ ID NO: 201, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   G) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 180, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 199, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein Xis D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   H) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 180, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 198, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   I) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 179, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein Xis D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   J) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 179, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 199, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein Xis D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   K) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 179, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 198, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   L) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 181, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   M) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 181, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 197, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 201, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   N) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 181, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises a VH domain sequence having at least 90%, 91%, 92%, 93%, 94%, 95%, 96%, 97%, 98%, 99%, or 100% sequence identity to the amino acid sequence of SEQ ID NO: 199, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   O) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 181, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 198, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R;
   or
   P) i) said first antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 178, wherein the VH domain comprises (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and a VL domain sequence having at least 90%, 91%, 92%, 93%, 94%, 95%, 96%, 97%, 98%, 99%, or 100% sequence identity to the amino acid sequence of SEQ ID NO: 182, wherein the VL domain comprises (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169; and
      ii) said second antigen-binding site comprises
      a VH domain sequence having at least 90%, 91%, 92%, 93%, 94%, 95%, 96%, 97%, 98%, 99%, or 100% sequence identity to the amino acid sequence of SEQ ID NO: 199, wherein the VH domain comprises (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and a VL domain sequence having at least 90%, 91%, 92%, 93%, 94%, 95%, 96%, 97%, 98%, 99%, or 100% sequence identity to the amino acid sequence of SEQ ID NO: 200, wherein the VL domain comprises (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R.
In certain embodiments of the invention, the bispecific antibody described herein binds to the first and second epitope on human CCL2 in ion-dependent manner.
In certain embodiments of the invention, the bispecific antibody described herein binds to human CCL2 in pH dependent manner and wherein the first antigen binding site and the second antigen binding site both bind to CCL2 with a higher affinity at neutral pH than at acidic pH.
In certain embodiments of the invention, the bispecific antibody described herein binds to human CCL2 with a 10 times higher affinity at pH 7.4, than at pH 5.8.
In certain embodiments of the invention, the bispecific antibody is an (isolated) (monospecific) antibody that (specifically) binds to a human CCL2, wherein the antibody comprises
   A) a VH domain comprising (a) a CDR-H1 comprising the amino acid sequence SHYGXS of SEQ ID NO: 164, wherein X is I or T, (b) a CDR-H2 comprising the amino acid sequence GX¹IX²IFX³TANYAQKFQG of SEQ ID NO: 165, wherein X¹ is V, I, or H, X² is P or H, and X³ is H or G, and (c) a CDR-H3 comprising the amino acid sequence YDAHYGELDF of SEQ ID NO: 166;
      and
      a VL domain comprising (d) a CDR-L1 comprising the amino acid sequence RASQHVSDAYLA of SEQ ID NO: 167; (e) a CDR-L2 comprising the amino acid sequence DASDRAE of SEQ ID NO: 168, and (f) a CDR-L3 comprising the amino acid sequence HQYIHLHSFT of SEQ ID NO: 169;
      or
   B)) a VH domain comprising (a) a CDR-H1 comprising the amino acid sequence HTYMH of SEQ ID NO: 183, (b) a CDR-H2 comprising the amino acid sequence RIDPXNHNTKFDPKFQG of SEQ ID NO: 184, wherein X is D or E, and (c) a CDR-H3 comprising the amino acid sequences GVFGFFXH of SEQ ID NO: 185, wherein X is D or E;
      and
      a VL domain comprising (d) a CDR-L1 comprising the amino acid sequence KAX¹EDIYNRX²A of SEQ ID NO: 186, wherein X¹ is F or T and X² is R or L, (e) a CDR-L2 comprising the amino acid sequence GATSLEH of SEQ ID NO: 187, and (f) a CDR-L3 comprising the amino acid sequence QQFXSAPYT of SEQ ID NO: 188, wherein X is W or R.

The term “epitope” includes any polypeptide determinant capable of specific binding to an antibody. In certain embodiments of the invention, epitope determinant includes chemically active surface groupings of molecules such as amino acids, sugar side chains, phosphoryl, or sulfonyl, and, in certain embodiments of the invention, may have specific three-dimensional structural characteristics, and or specific charge characteristics. An epitope is a region of an antigen that is bound by an antibody.

One can easily determine whether an antibody binds to the same epitope as, or competes for binding with, a reference anti-CCL2 antigen binding site by using routine methods known in the art. For example, to determine if a test antibody binds to the same epitope as a reference anti-CCL2 antigen binding site of the invention, the reference antibody is allowed to bind to CCL2 domain thereof under saturating conditions. Next, the ability of a test antibody to bind to human CCL2 is assessed. If the test antibody is able to bind to human CCL2 following saturation binding with the reference anti-CCL2 antigen binding site, it can be concluded that the test antibody binds to a different epitope than the reference anti-CCL2 antigen binding site. On the other hand, if the test antibody is not able to bind to human CCL2 following saturation binding with the reference anti-CCL2 antibody, then the test antibody may bind to the same epitope as the epitope bound by the reference anti-CCL2 antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance (e.g. BIAcore), flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502).

Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding with a reference anti-CCL2 antibody, the above-described binding methodology is performed in two orientations: In a first orientation, the reference antibody is allowed to bind to CCL2 under saturating conditions followed by assessment of binding of the test antibody to human CCL2. In a second orientation, the test antibody is allowed to bind to a CCL2 molecule under saturating conditions followed by assessment of binding of the reference antibody to humans CCL2. If, in both orientations, only the first (saturating) antibody is capable of binding to the CCL2 molecule, then it is concluded that the test antibody and the reference antibody compete for binding to CCL2. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

When used herein, the term “CCL2”, “human CCL2”, which also called “MCP-1” is meant the 76 amino acid sequence referenced in NCBI record accession No. NP_002973 (SEQ ID NO: 117) and variously known as CCL2, MCP-1 (monocyte chemotactic protein 1), SMC-CF (smooth muscle cell chemotactic factor), LDCF (lymphocyte-derived chemotactic factor), GDCF (glioma-derived monocyte chemotactic factor), TDCF (tumor-derived chemotactic factors), HCl1 (human cytokine 11), MCAF (monocyte chemotactic and activating factor). The gene symbol is SCYA2, the JE gene on human chromosome 17, and the new designation is CCL2 (Zlotnik, Yoshie 2000. Immunity 12: 121-127). JE is the mouse homolog of human MCP- 1/CCL2.

Handel and others (Biochemistry. 1996; 35: 17269-6584) determined the solution structure of a CCL2 dimer. These studies indicated that the secondary structure of CCL2 consists of four β-sheets. Additionally, the residues responsible for the dimerization interface of CCL2 were described by Zhang and Rollins (Mol Cell Biol. 1995; 15: 15751-4855). The protein complex appears elongated with the two monomers oriented in such a way that they form a large pocket. Structures of monomeric and dimeric CCL2 in two crystal forms, the so-called I and P forms, have also been determined (Lubkowski et al., Nat Struct Biol. 1997; 4: 171-69). Paolini et al, (J. Immuno1.1994 Sep 15;153(6):2704-17), described that MCP1/CCL2 exists as a monomer at physiologically relevant concentrations: By analyzing rec. CCL2 protein (purchased from Peprotech) with size exclusion HPLC, sedimentation equilibrium ultracentrifugation and chemical cross-linking, they could show that the weight fraction of monomeric and dimeric forms of MCP-1 depends on the concertation in vitro. Finally, Seo and colleagues (J. Am. Chem. Soc. 2013 Mar 20;135(11):4325-32) could show by ion mobility mass spectrometry the presence of injected CCL2 in both monomeric and dimeric forms under physiological conditions.

Thus “wild type CCL-2” (wt CCL2) can exist as monomer but actually can also form dimers at physiological concentrations. This monomer-dimer equilibrium is certainly different and has to be carefully taken into account for all in vitro experiments described where different concentrations might be used. To avoid any uncertainties, we generated point mutated CCL2 variants: The “P8A” variant of CCL2 carries a mutation in the dimerization interface resulting in an inability to form a dimer leading to a defined, pure CCL2 monomer. In contrast, the “T10C”“variant of CCL2 results in a fixed dimer of CCL2 (J. Am. Chem. Soc. 2013 Mar 20;135(11):4325-32).

The CCL2/CCR2 axis is the main mediator of immature myeloid cell recruitment into the tumor. CCL2 is overexpressed by malignant cells and binds to the extracellular matrix (ECM) building up a chemoattractant gradient. Once they reach the tumor, myeloid-derived suppressive cells (MDSCs) contribute to the pro-tumorigenic milieu by secreting/up-regulating anti-inflammatory cytokines/receptors that in turn inhibit the initiation of an anti-tumor T cell response. In this way, MDSCs may reduce or even impair the efficacy of any T cell-activating therapy (Meyer et al, 2014). Therefore, the specific inhibition of the recruitment of these immature myeloid cells will boost the efficacy of checkpoint inhibitors, T cell bispecific and cancer immune therapies. In addition, CCL2 has also been implicated in the promotion of angiogenesis, metastasis and tumor growth, suggesting that neutralizing CCL2 might contribute to several lines of anti-tumor intervention.

Targeting CCL2—as opposed to its receptor—will specifically inhibit the undesired CCL2-mediated effects, sparing those that might signal through the same receptor (CCR2) but different ligands (e.g. CCL7, CCL8, CCL13) which are involved in the recruitment of other immune cell populations, like Th1 and NK cells.

Clinically, CCL2 has been a preferred antibody-target in several studies aiming at neutralizing its elevated levels caused by different inflammatory diseases, such as rheumatoid arthritis (Haringman et al, 2006), idiopathic pulmonary fibrosis (Raghu et al, 2015), diabetic nephropathy (Menne et al, 2016) and cancer (Sandhu et al, 2013). However, its high synthesis rate together with the observed high in vivo antibody-antigen dissociation constants (KD) have proven to be the main obstacles hindering the suppression of free CCL2 by conventional antibodies at clinically viable doses (Fetterly et al, 2013).

CCL2 neutralization appears to be more obviously relevant in patients with elevated serum levels of CCL2, which has been observed in several types of cancers like breast cancer (BC), ovarian cancer (OvCa), colorectal cancer (CRC), pancreatic cancer and prostate cancer. However, even patients within these indications who do not present this serology but whose tumors are highly infiltrated with immune cells of the myeloid lineage might very well profit from this novel therapy due to the many roles that CCL2 plays in the tumor context as mentioned above.

As used herein, an antibody “binding to human CCL2”, “specifically binding to human CCL2”, “that binds to human CCL2” or “anti-CCL2 antibody” refers to an antibody specifically binding to the human CCL2 antigen with a binding affinity of a KD-value of 5.0×10⁻⁸ mol/l or lower, in certain embodiments of a K_{D}-value of 1.0×10⁻⁹ mol/l or lower, in certain embodiments of a K_{D}-value of 5.0×10⁻⁸ mol/l to 1.0×10⁻¹³ mol/l.

The binding affinity is determined with a standard binding assay, such as surface plasmon resonance technique (BIAcore®, GE-Healthcare Uppsala, Sweden) e.g. using constructs comprising CCL2 extracellular domain (e.g. in its natural occurring 3 dimensional structure). In certain embodiments of the invention, binding affinity is determined with a standard binding assay using exemplary soluble CCL2.

Antibody specificity refers to selective recognition of the antibody for a particular epitope of an antigen. Natural antibodies, for example, are monospecific.

The term “bispecific antibody that binds to (human) CCL2”, “biparatopic antibody that binds to (human) CCL2”, “bispecific anti-CCL2 antibody”, “biparatopic anti-CCL2 antibody” as used herein means that the antibody is able to specifically bind to at least two different epitopes on (human) CCL2. Typically, such bispecific antibody comprises two different antigen binding sites (two different paratopes), each of which is specific for a different epitope of (human) CCL2. In certain embodiments of the invention, the bispecific antibody is capable of binding two different and non-overlapping epitopes on CCL2, which means that the two different antigen binding sites do not compete for binding to CCL2.

As used herein, the term “antigenic determinant” or “antigen” refers to a site on a polypeptide macromolecule to which an antigen binding moiety/site binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

The following exemplification of the method according to the invention is presented using an exemplary anti-CS antibody. This is presented as mere exemplification and shall not be construed as limitation of the method according to the current invention. The true scope is set forth in the appended claims.

The exemplification shows the in vitro determination of free human C5 in 100% human serum samples.

To detect free C5 in human serum samples a nanoliter-scale, microfluidic, affinity flow-through format with laser-induced fluorescence detection assay (Gyrolab® workstation assay) was used. With the method quantitative detection of free C5 in 100% human serum is possible.

Test samples, quality control samples and positive control standards are analyzed in 100% serum. Quality control samples and standards are prepared in 100% horse serum (non-cross reactive C5). Relative quantification of the analyte is performed by back-calculation of the fluorescence values using the corresponding standard curve e.g. with a non-linear 4-parameter Wiemer-Rodbard fitting function.

A typical calibration curve is shown in FIG. 5 (Examples 1 and 10).

In certain embodiments of the invention, the therapeutic antibody is an anti-C5 antibody and the antigen is human C5.

The terms “anti-C5 antibody” and “an antibody that (specifically) binds to C5” refer to an antibody that is capable of binding C5 with sufficient affinity such that the antibody is useful as a diagnostic and/or therapeutic agent in targeting C5. In certain embodiments of the invention, the extent of binding of an anti-C5 antibody to an unrelated, non-C5 protein is less than about 10% of the binding of the antibody to C5. In certain embodiments of the invention, an anti-C5 antibody binds to an epitope of C5 that is conserved among C5 from different species. In one preferred embodiment, C5 is human C5.

In certain embodiments of the invention, the anti-C5 antibody is Eculizumab or Crovalimab.

In certain embodiments of the invention, the determination of free C5 is in the presence of Eculizumab and/or Crovalimab. In this embodiment, the term “free C5” denotes C5 of any length but neither bound by Eculizumab nor Crovalimab.

The term “C5”, as used herein, encompasses any native C5 from any vertebrate source, including mammals such as primates (e.g., humans and monkeys) and rodents (e.g., mice and rats). Unless otherwise indicated, the term “C5” refers to a human C5 protein having the amino acid sequence shown in SEQ ID NO: 30 and containing the beta chain sequence shown in SEQ ID NO: 31. The term encompasses “full-length”, unprocessed C5 as well as any form of C5 that results from processing in the cell. The term also encompasses naturally occurring variants of C5, e.g., splice variants or allelic variants. The amino acid sequence of an exemplary human C5 is shown in SEQ ID NO: 30 (“wild-type” or “wt” C5). The amino acid sequence of an exemplary beta chain of human C5 is shown in SEQ ID NO: 31. The amino acid sequences of exemplary MG1, MG2 and MG1-MG2 domains of the beta chain of human C5 are shown in SEQ ID NO: 32, 33, and 34, respectively. The amino acid sequences of exemplary cynomolgus monkey and murine C5 are shown in SEQ ID NO: 35 and 96, respectively. Amino acid residues 1-19 of SEQ ID NOs: 30, 31, 34, and 96 correspond to a signal sequence that is removed during processing in the cell and is thus missing from the corresponding exemplary amino acid sequence.

US 2016/0167054 discloses anti-C5 antibodies and methods of using the same. In some embodiments, an isolated anti-C5 antibody disclosed binds to an epitope within the beta chain of C5 with a higher affinity at neutral pH than at acidic pH.

C5 is a 181 kDa protein found in normal serum at approximately 71 μg/ml (0.4 μM). C5 is glycosylated with about 1.5-3% of its mass attributed to carbohydrate. Mature C5 is a heterodimer of 106 kDa alpha chain that is disulfide linked to 66 kDa beta chain. C5 is synthesized as a single chain precursor protein (pro-C5 precursor) of 1577 amino acids (see, e.g., U.S. Pat. Nos. 6,355,245 and 7,432,356). The pro-C5 precursor is cleaved to yield the beta chain as an amino terminal fragment and the a chain as alpha carboxyl terminal fragment. The alpha chain and the beta chain polypeptide fragments are connected to each other via a disulfide bond and constitute the mature C5 protein.

Mature C5 is cleaved into the C5a and C5b fragments during activation of the complement pathways. C5a is cleaved from the alpha chain of C5 by C5 convertase as an amino terminal fragment comprising the first 65 amino acids of the alpha chain. The remaining portion of mature C5 is fragment C5b, which contains the rest of the alpha chain disulfide bonded to the beta chain. Approximately 20% of the 11 kDa mass of C5a is attributed to carbohydrate.

C5a is an anaphylatoxin. C5b combines with C6, C7, C8 and C9 to form the membrane attack complex (MAC, C5b-9, terminal complement complex (TCC)) at the surface of the target cell. When sufficient numbers of MACs are inserted into target cell membranes, MAC pores are formed to mediate rapid osmotic lysis of the target cells.

Anaphylatoxins can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract granulocytes such as neutrophils, eosinophils, basophils and monocytes to the site of complement activation.

The activity of C5a is regulated by the plasma enzyme carboxypeptidase N that removes the carboxy-terminal arginine from C5a forming C5a-des-Arg derivative. C5a-des-Arg exhibits only 1% of the anaphylactic activity and polymorpho nuclear chemotactic activity of unmodified C5a.

While a properly functioning complement system provides a robust defense against infecting microbes, inappropriate regulation or activation of complement has been implicated in the pathogenesis of a variety of disorders including, e.g., rheumatoid arthritis (RA); lupus nephritis; ischemia-reperfusion injury; paroxysmal nocturnal hemoglobinuria (PNH); atypical hemolytic uremic syndrome (aHUS); dense deposit disease (DDD); macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis (see, e.g., Holers et al., Immunol. Rev. 223 (2008) 300-316). Therefore, inhibition of excessive or uncontrolled activations of the complement cascade can provide clinical benefits to patients with such disorders, especially to patients with Paroxysmal nocturnal hemoglobinuria (PNH).

Eculizumab is a humanized monoclonal antibody directed against the complement protein C5, and the first therapy approved for the treatment of paroxysmal nocturnal hemoglobinuria (PNH) and atypical hemolytic uremic syndrome (aHUS) (see, e.g., Dmytrijuk et al., The Oncologist 13 (2008) 894-910). Eculizumab inhibits the cleavage of C5 into C5a and C5b by C5 convertase, which prevents the generation of the terminal complement complex C5b-9. Both C5a and C5b-9 cause the terminal complement-mediated events that are characteristic of PNH and aHUS (see also, WO 2005/065607, WO 2007/96586, WO 2008/060790, and WO 2010/054403). Several reports have described other anti-C5 antibodies. For example, WO 86/28707 described an anti-C5 antibody that binds to the alpha chain of C5 but does not bind to C5a, and blocks the activation of C5, while WO 2002/30886 described an anti-C5 monoclonal antibody that inhibits C5a formation. On the other hand, WO 2004/006653 described an anti-C5 antibody that recognizes the proteolytic site for C5 convertase on the alpha chain of C5, and inhibits the conversion of C5 to C5a and C5b. WO 2010/015608 described an anti-C5 antibody that has an affinity constant of at least 1×10E7 M−1. In certain embodiments of the invention, the drug is Eculizumab.

In some embodiments, the anti-C5 antibody is binding to an epitope within the beta chain of C5. In some embodiments, the anti-C5 antibody binds to an epitope within the MG1-MG2 domain of the beta chain of C5. In some embodiments, the anti-C5 antibody binds to an epitope within a fragment consisting of amino acids 27-115 of the beta chain (SEQ ID NO: 31) of C5. In some embodiments, the anti-C5 antibody binds to an epitope within the beta chain (SEQ ID NO: 31) of C5 which comprises at least one fragment selected from the group consisting of amino acids 38-48, 61-67, and 98-101. In some embodiments, the anti-C5 antibody binds to an epitope within a fragment of the beta chain (SEQ ID NO: 31) of C5 which comprises at least one amino acid residue selected from the group consisting of Glu48, Asp51, His61, His63, Lys100, and His101 of SEQ ID NO: 31. In further embodiments, the antibody binds to C5 with a higher affinity at neutral pH than at acidic pH. In further embodiments, the antibody binds to C5 with a higher affinity at pH 7.4 than at pH 5.8. In another embodiment, the anti-C5 antibody binds to the same epitope as an antibody described in Table 1. In further embodiments, the antibody binds to the same epitope as an antibody described in Table 1 with a higher affinity at pH 7.4 than at pH 5.8. In a further embodiment, the anti-C5 antibody binds to the same epitope as an antibody described in Tables 2 or 3. In further embodiments, the antibody binds to the same epitope as an antibody described in Tables 2 or 3 with a higher affinity at pH 7.4 than at pH 5.8.

TABLE 1
SEQ ID NO:

| | | | HVR- | HVR- | HVR- | HVR- | HVR- | HVR- |
|---|---|---|---|---|---|---|---|---|
| antibody | VH | VL | H1 | H2 | H3 | L1 | L2 | L3 |
| CFA0305 | 1 | 11 | 36 | 46 | 56 | 66 | 76 | 86 |
| CFA0307 | 2 | 12 | 37 | 47 | 57 | 67 | 77 | 87 |
| CFA0357 | 3 | 13 | 38 | 48 | 58 | 68 | 78 | 88 |
| CFA0501 | 4 | 14 | 39 | 49 | 59 | 69 | 79 | 89 |
| CFA0538 | 5 | 15 | 40 | 50 | 60 | 70 | 80 | 90 |
| CFA0590 | 6 | 16 | 41 | 51 | 61 | 71 | 81 | 91 |
| CFA0567 | 7 | 17 | 42 | 52 | 62 | 72 | 82 | 92 |
| CFA0573 | 8 | 18 | 43 | 53 | 63 | 73 | 83 | 93 |
| CFA0576 | 9 | 19 | 44 | 54 | 64 | 74 | 84 | 94 |

TABLE 2
SEQ ID NO:

| antibody | VH | HVR-H1 | HVR-H2 | HVR-H3 |
|---|---|---|---|---|
| 305L05 | 10 | 45 | 55 | 65 |
| 305L015 | 97 | 108 | 109 | 112 |
| 305L016 | 98 | 108 | 110 | 112 |
| 305L018 | 99 | 108 | 109 | 112 |
| 305L019 | 100 | 108 | 109 | 112 |
| 305L020 | 100 | 108 | 109 | 112 |
| 305L022 | 100 | 108 | 109 | 112 |
| 305L023 | 101 | 108 | 111 | 112 |

TABLE 3
SEQ ID NO:

| antibody | VL | HVR-L1 | HVR-L2 | HVR-L3 |
|---|---|---|---|---|
| 305L05 | 20 | 75 | 85 | 95 |
| 305L015 | 102 | 113 | 114 | 116 |
| 305L016 | 102 | 113 | 114 | 116 |
| 305L018 | 102 | 113 | 114 | 116 |
| 305L019 | 102 | 113 | 114 | 116 |
| 305L020 | 103 | 113 | 114 | 116 |
| 305L022 | 104 | 113 | 115 | 116 |
| 305L023 | 104 | 113 | 115 | 116 |

In certain embodiments of the invention, the anti-C5 antibody competes for binding to C5 with an antibody comprising a VH and VL pair selected from: (a) a VH of SEQ ID NO: 01 and a VL of SEQ ID NO: 11; (b) a VH of SEQ ID NO: 05 and a VL of SEQ ID NO: 15; (c) a VH of SEQ ID NO: 04 and a VL of SEQ ID NO: 14; (d) a VH of SEQ ID NO: 06 and a VL of SEQ ID NO: 16; (e) a VH of SEQ ID NO: 02 and a VL of SEQ ID NO: 12; (f) a VH of SEQ ID NO: 03 and a VL of SEQ ID NO: 13; (g) a VH of SEQ ID NO: 09 and a VL of SEQ ID NO: 19; (h) a VH of SEQ ID NO: 07 and a VL of SEQ ID NO: 17; (i) a VH of SEQ ID NO: 08 and a VL of SEQ ID NO: 18; and (j) a VH of SEQ ID NO: 10 and a VL of SEQ ID NO: 20.

In certain embodiments of the invention, the anti-CS antibody is used in treating a complement-mediated disease or condition that involves excessive or uncontrolled activation of C5. In additional embodiments, the anti-C5 antibody is used in treating diseases or disorders that include but are not limited to, paroxysmal nocturnal hemoglobinuria (PNH), age-related macular degeneration, myocardial infarction, rheumatoid arthritis, osteoporosis, osteoarthritis, and inflammation. The anti-C5 antibody is used to enhance the clearance of C5 from plasma.

In certain embodiments of the invention, the method is for the detection of free C5 in the presence of an anti-C5 antibody comprising a VH as in any of the embodiments provided above and a heavy chain constant region comprising the amino acid sequence of any one of SEQ ID NOs: 27, 28, 29, 105, 106, and 107. In certain embodiments of the invention, the method is for the detection of free C5 in the presence of an anti-C5 antibody comprising a VL as in any of the embodiments provided above and a light chain constant region comprising the amino acid sequence of any one of SEQ ID NOs: 36, 37, and 38.

In certain embodiments of the invention, the method is for the detection of free C5 in the presence of an anti-C5 antibody that competes for binding to C5 with an antibody comprising a VH and VL pair selected from: (a) a VH of SEQ ID NO: 01 and a VL of SEQ ID NO: 11; (b) a VH of SEQ ID NO: 22 and a VL of SEQ ID NO: 25; (c) a VH of SEQ ID NO: 21 and a VL of SEQ ID NO: 24; (d) a VH of SEQ ID NO: 05 and a VL of SEQ ID NO: 15; (e) a VH of SEQ ID NO: 04 and a VL of SEQ ID NO: 14; (f) a VH of SEQ ID NO: 06 and a VL of SEQ ID NO: 16; (g) a VH of SEQ ID NO: 02 and a VL of SEQ ID NO: 12; (h) a VH of SEQ ID NO: 03 and a VL of SEQ ID NO: 13; (i) a VH of SEQ ID NO: 09 and a VL of SEQ ID NO: 19; (j) a VH of SEQ ID NO: 7 and a VL of SEQ ID NO: 17; (k) a VH of SEQ ID NO: 8 and a VL of SEQ ID NO: 18; (l) a VH of SEQ ID NO: 23 and a VL of SEQ ID NO: 26; and (m) a VH of SEQ ID NO: 10 and a VL of SEQ ID NO: 20.

In certain embodiments of the invention, the method is for the detection of free C5 in the presence of an anti-C5 antibody that competes for binding C5 with an antibody comprising a VH and VL pair selected from: (a) a VH of SEQ ID NO: 22 and a VL of SEQ ID NO: 25; (b) a VH of SEQ ID NO: 21 and a VL of SEQ ID NO: 24; (c) a VH of SEQ ID NO: 05 and a VL of SEQ ID NO: 15; (d) a VH of SEQ ID NO: 04 and a VL of SEQ ID NO: 14; (e) a VH of SEQ ID NO: 06 and a VL of SEQ ID NO: 16; (f) a VH of SEQ ID NO: 02 and a VL of SEQ ID NO: 12; (g) a VH of SEQ ID NO: 03 and a VL of SEQ ID NO: 13; (h) a VH of SEQ ID NO: 09 and a VL of SEQ ID NO: 19; (i) a VH of SEQ ID NO: 07 and a VL of SEQ ID NO: 17; (j) a VH of SEQ ID NO: 8 and a VL of SEQ ID NO: 18; (k) a VH of SEQ ID NO: 23 and a VL of SEQ ID NO: 26.

In one preferred embodiment of the invention, the method is for the detection of free C5 in the presence of an anti-C5 antibody comprising a VH of SEQ ID NO: 97 and a VL of SEQ ID NOs: 102.

The following examples, sequences and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1A cynomolgus CCL2 calibration curve was prepared with an assay according to the invention in 100% horse serum in a range of 4 to 1000 pg/mL CCL2 serum concentration and analyzed in an Elisa Assay as described in Example 4 including a variation of the incubation time of the sample on the assay plate between 75 seconds and 12 minutes.

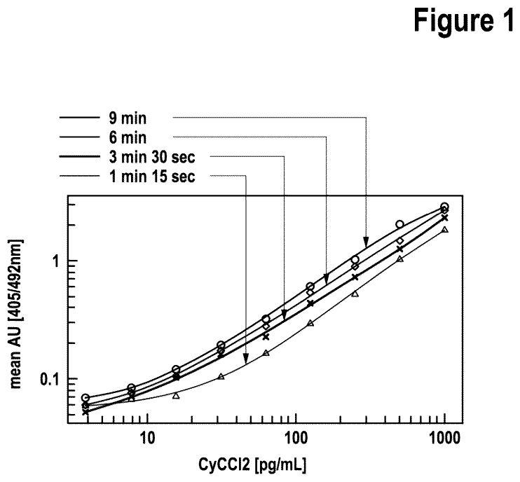

FIG. 2 Nanoliter-scale, microfluidic, affinity flow-through format with laser-induced fluorescence detection assay with indirect and direct Alexa labelling of the detection-antibody according to the invention.

FIG. 3 Nanoliter-scale, microfluidic, affinity flow-through format with laser-induced fluorescence detection assay according to the invention with therapeutic antibody and competitive rabbit monoclonal antibodies were used. CCL2 values were back-calculated on the calibration curve and CCL2 recovery (% free) was calculated relative to the non-spiked 5 ng/mL CCL2 value; upper curve with therapeutic antibody as capture antibody and lower curve with competitive rabbit antibody as capture antibody.

FIG. 4 Assay according to the invention performed with recombinant human wild-type CCL2 as calibrator. Calibration range of two runs is shown.

FIG. 5 Calibration curve of an assay according to the invention to detect free C5 in human serum samples a nanoliter-scale, microfluidic, affinity flow-through format with laser-induced fluorescence detection assay (Gyrolab® workstation assay).

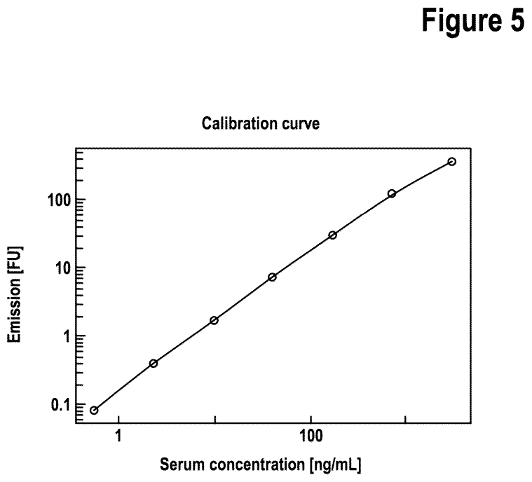

FIG. 6 Scheme of the method according to the invention using an ELISA.

FIG. 7 Scheme of comparative ELISA in 25% serum (Example 4).

FIG. 8 Scheme of the method according to the invention using a nanoliter-scale, microfluidic, affinity flow-through format with laser-induced fluorescence detection, indirect format (Gyros assay) (Examples 6, 7).

FIG. 9 Calibration curve of the nanoliter-scale, microfluidic, affinity flow-through format with laser-induced fluorescence detection (Gyros assay) (Example 6).

FIG. 10 Scheme of the method according to the invention using a nanoliter-scale, microfluidic, affinity flow-through format with laser-induced fluorescence detection, direct format (Gyros assay) (Example 7).

FIG. 11 Scheme of the method according to the invention using a nanoliter-scale, microfluidic, affinity flow-through format with laser-induced fluorescence detection (Gyros assay) (Example 9).

### EXAMPLE 1

### General Description of the Method According to the Invention

### 1) Nanoliter-Scale, Microfluidic, Affinity Flow-Through Format with Laser-Induced Fluorescence Detection Based Assay Format

To detect free antigen in human serum samples a Gyrolab® workstation assay was set-up. The test was used for quantitative detection of free antigen. Test samples, quality control samples and positive control standards were analyzed in 100% serum. Quality control samples and standards were prepared in 100% horse serum (comprising non-cross reactive endogenous target).

The following consecutive steps were performed:

Addition of capture reagent (mAb<target>rH-IgG-Bi, 5,000 ng/mL) followed by addition of sample and finally detection reagent (mAb<target>M-Alexa 647, 10,000 ng/mL). After each step a washing was performed to remove non-bound reagents. Each wash step consisted of the addition of a wash solution (1× PBS with 0.05% (v/v) Tween 20).

### 2) ELISA-Based Assay Format

To detect free antigen in human serum samples an ELISA-assay is set-up. The test is used for quantitative detection of free antigen. Test samples, quality control samples and positive control standards are analyzed in 100% serum. Quality control samples and standards are prepared in 100% horse serum (comprising non-cross reactive endogenous target).

The following consecutive steps are performed:

Different combinations of recombinant antigen and antibody are prepared in 100% horse pooled serum and incubated for 2 hours at RT. The calibration curve samples are prepared in 100% horse serum. Briefly, biotinylated (bispecific) therapeutic antibody as capture antibody, test sample and detection reagent (digoxygenylated (bispecific) therapeutic antibody are added stepwise to a 384-well streptavidin-coated microtiter plate and incubated on a non-vigorous shaker for 3-4 minutes (3.5 minutes as target). For detection of immobilized immune complexes, a polyclonal anti-digoxygenin-POD conjugate is added and the plate is incubated for 15-20 minutes. Optionally, the plate is washed three times after each step to remove unbound substances. ABTS is added to the plate and incubated at room temperature with shaking. Absorption is measured at 405/490 nm wavelength. The antigen concentrations are calculated based on the response of the calibration curve using the analytical software XLfit (IDBS).

### EXAMPLE 2

### Determination of KD Value in 100% Serum Sample

Based on the general method as described in Example 1 the following experiment was set up with the purpose to determine the KD value of a monoclonal antibody against its endogenous antigen. The calibration curve covered the range of 0.55 ng/mL to 3000 ng/mL. QC samples were prepared in horse serum at 5 concentrations. 0.55 ng/mL, 1.5 ng/mL, 100 ng/mL, 1200 ng/mL and 3000 ng/mL. Recovery of the QC samples met the criterion of +/−20% from the nominal value and were in the range of 97% to 112%. For determination of the KD value (following the method as outlined in WO 2014/023655) to endogenous target three human serum samples were diluted by a factor of 40 with horse serum to maintain 100% serum concentration and spiked with 1000 ng/mL, 2000 ng/mL, 3000 ng/mL, 4000 ng/mL and 5000 ng/mL of therapeutic antibody and equilibrated. A non-spiked human serum sample was analyzed with a dilution factor of 40 (with horse serum) as well to calculate the total target concentration in each individual serum sample. Based on the determined total target value, free target fraction was determined for each spiked serum sample. Based on each free fraction the KD value was calculated.

| mAb<target> | ID 1 | ID 2 | ID 3 |
|---|---|---|---|
| concentration [ng/ml] | KD [nM] | KD [nM] | KD [nM] |
| 5000 | 0.049 | 0.076 | 0.048 |
| 4000 | 0.046 | 0.043 | 0.051 |
| 3000 | 0.056 | 0.041 | 0.042 |
| 2000 | 0.036 | 0.040 | 0.039 |
| 1000 | 0.046 | 0.072 | 0.060 |
| average = | 0.047 | 0.054 | 0.048 |
| CV = | 15% | 34% | 17% |

The average (av) KD was 0.05 nM with a standard deviation (cv) of 0.012 nM.

### EXAMPLE 3

### Assay According to the Invention in 100% Human Serum

Based on Example 2 the following experiment was set up with the focus on the preparation and analysis of Free QC samples in 100% human serum. Free QC samples were prepared by addition of 100 μg/mL Crovalimab to each in Example 2 used individual human serum. Each serum sample was analyzed undiluted and free target concentration were analyzed as following: ID1=6.5 ng/mL; ID2=8.2 ng/mL; and ID3=7.6 ng/mL. Based on the KD value and total target concentration determined in Example 2 the estimated free target concentration should be in the range for ID1 of 2.41 ng/mL to 6.83 ng/mL, for ID2 in the range of 3.38 ng/mL to 9.56 ng/mL and for ID3 in the range of 3.51 ng/mL to 9.93 ng/mL. Calculation of the range is based on 2-fold standard deviation of the KD value. All samples of the three individual serum sample were within the calculated range, thus, verifying the correctness of the method according to the invention.

### EXAMPLE 4

### Comparative Example: ELISA in 25% Serum

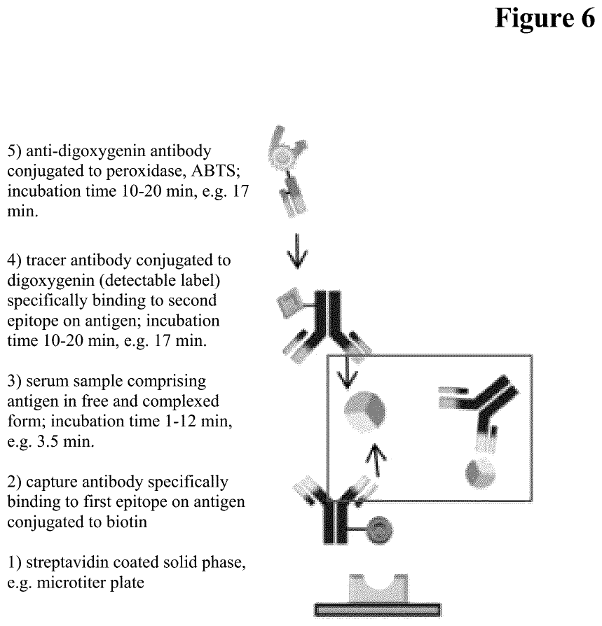

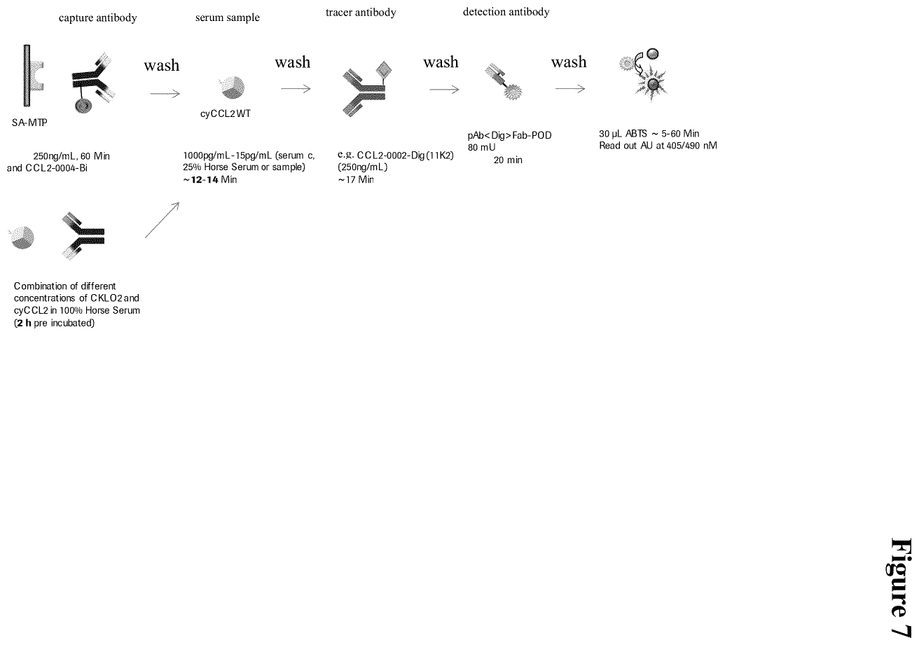

Different combinations of recombinant cynomolgus CCL2 and biparatopic anti-CCL2 antibody CKLO2-SG1 were prepared in 100% horse pooled serum and incubated for 2 hours at RT. Samples were diluted 1 to 4 (MRD, resulting in 25% matrix) in assay buffer (PBS, 0.1% Tween, 1% BSA) and additionally 1 to 10 diluted in assay buffer containing 25% horse serum. The calibration curve was prepared in assay buffer containing 25% horse serum and covered the range of 7.8 to 1000 pg/mL CCL2 serum concentration. Briefly, biotinylated anti-CCL2 capture antibody (CNTO0888, CCL2-0004), test sample and detection reagent (digoxygenylated anti-CCL2 antibody (Humanized 11K2, CCL2-0002)), were added stepwise to a 384-well streptavidin-coated microtiter plate and incubated on a non-vigorous shaker for 1 hour, 12 to 14 minutes and 17 minutes respectively. For detection of immobilized immune complexes, a polyclonal anti-digoxygenin-POD conjugate was added and the plate was incubated for 20 minutes. The plate was washed three times after each step to remove unbound substances. ABTS was added to the plate and incubated at room temperature with shaking. Absorption was measured at 405/490 nm wavelength (see FIGS. 6 and 7). The CCL2 concentrations were calculated based on the response of the calibration curve using the analytical software XLfit (IDBS) including the dilution factors 1 to 4 and 1 to 40. A ratio of the two analyzed sample dilutions was calculated and is shown in the following Table (-=not determined)

| ng/ml | Ratio of determined CCL2 value dilution 1 to 40/ | | | | | | | | | |
|---|---|---|---|---|---|---|---|---|---|---|
| cyCCL2 | Measured CCL2 value dilution 1 to 4 | | | | | | | | | |
| 500.00 | 10.9 | 8.2 | — | — | — | — | — | — | — | |
| 100.00 | 12.5 | 8.8 | 6.6 | — | — | — | — | — | — | |
| 20.00 | — | 8.4 | 6.2 | 5.2 | — | — | — | — | — | |
| 4.00 | — | — | 6.3 | 5.0 | 3.0 | — | — | — | — | |
| 0.80 | — | — | — | — | 3.1 | 1.8 | 1.3 | 1.1 | 1.1 | |
| 0.16 | — | — | — | — | — | — | — | — | — | |
| 0.032 | — | — | — | — | — | — | — | — | — | |
| 0 | — | — | — | — | — | — | — | — | — | |
| | 50000 | 12500 | 3125 | 781 | 195 | 49 | 12 | 3 | 0 | ng/mL |
| | | | | | | | | | | CKLO2-SG1 |

### EXAMPLE 5

### Assay According to the Invention in 100% Serum

A cynomolgus CCL2 calibration curve was prepared in 100% horse serum in a range of from 4 pg/mL to 1000 pg/mL CCL2 and analyzed in an ELISA format based on that as described in Example 4 but with the samples in 100% serum (no dilution). The incubation time of the sample on the assay plate was varied between 75 seconds and 12 minutes.

| | depicted mean AU values [n = 2] | | | | |
|---|---|---|---|---|---|
| | incubation time | | | | |
| pg/ml | 12 | 9 | 6 | 3 Min | 1 Min |
| cyCCL2 | Min | Min | Min | 30 s | 15 s |
| 1000.00 | 3.01 | 2.86 | 2.65 | 2.31 | 1.81 |
| 500.00 | 2.37 | 2.01 | 1.48 | 1.25 | 1.02 |
| 250.00 | 1.15 | 1.03 | 0.89 | 0.72 | 0.52 |
| 125.00 | 0.72 | 0.61 | 0.54 | 0.44 | 0.29 |
| 62.50 | 0.38 | 0.32 | 0.28 | 0.22 | 0.16 |
| 31.25 | 0.21 | 0.19 | 0.17 | 0.16 | 0.10 |
| 15.63 | 0.14 | 0.12 | 0.10 | 0.10 | 0.07 |
| 7.81 | 0.10 | 0.08 | 0.08 | 0.07 | 0.07 |
| 3.91 | 0.07 | 0.07 | 0.06 | 0.05 | 0.06 |
| 0 | 0.05 | 0.05 | 0.05 | 0.05 | 0.05 |

### EXAMPLE 6

### Method According to the Invention Using a Nanoliter-Scale, Microfluidic, Affinity Flow-Through Format with Laser-Induced Fluorescence Detection (Gyros Assay)

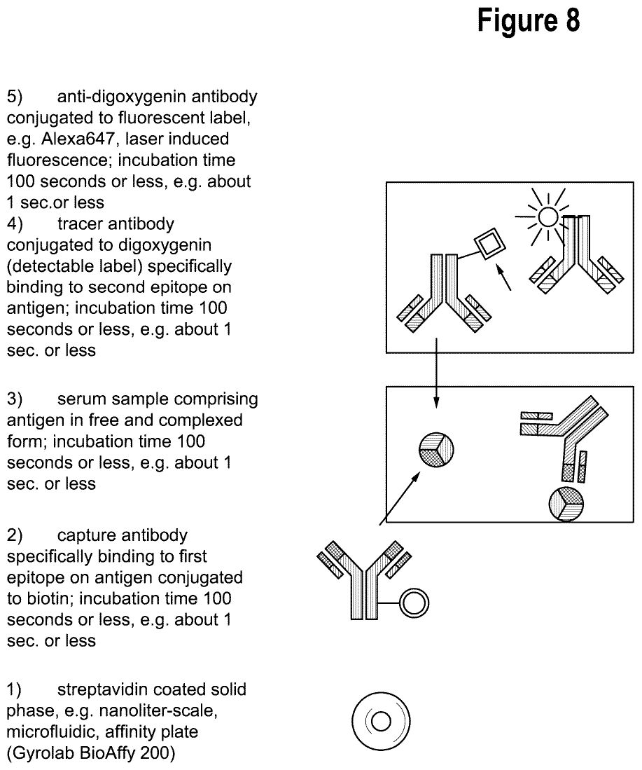

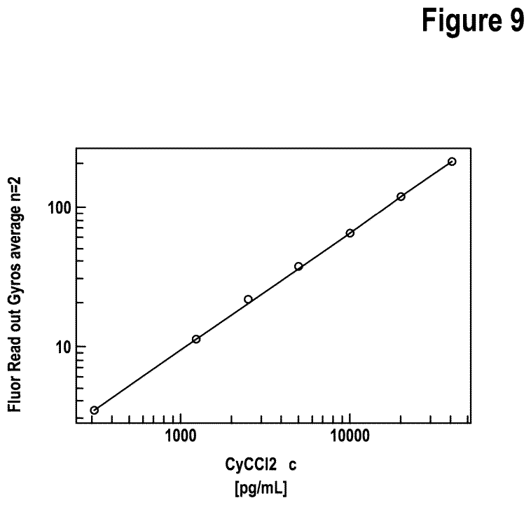

A Gyrolab™ based method was used in this example. Recombinant cynomolgus CCL2 samples were prepared in assay buffer (PBS, 0.1% (v/v) Tween-20, 1% BSA) and analyzed on a Gyrolab Xplore. The monospecific biotinylated parental anti-CCL2 antibody (CNT00888, alias CCL2-004) was used as capture reagent diluted to 1 μg/mL in assay buffer. For detection 1 μg/ml of the monospecific dig labeled anti-CCL2 antibody (Humanized 11K2, CCL2-0002) was pre-incubated with 1 μg/mL mAb<Dig>M-1.71.256-IgG-Alexa 647 for two hours in assay buffer. All reagents and samples were transferred to a 96-well PCR plate and loaded into the instrument together with a Gyrolab BioAffy 200 nL disc (Gyros Protein Technologies AB). A three-step assay protocol (200-3W-001) was selected. Briefly, the protocol describes the sequential addition of capture reagent, sample and detection reagent to designated streptavidin columns of the Gyrolab BioAffy 200 disc. Each reagent reaches the column at the same time after a short spinning step is applied to the disc. The columns were washed with PBS with 0.05 Tween after each step and finally laser induced fluorescence values were recorded within the instrument. A non-linear 4-parameter curve-fitting function (Wiemer-Rodbard) was applied to the averaged raw data to obtain a calibration curve. See also FIGS. 8 and 9.

| cyCCL2c | Fluorescence read out | | | standard- | coefficient | signal to |
|---|---|---|---|---|---|---|
| [pg/mL] | replicate 1 | replicate 2 | average | deviation | of variance | noise |
| 40000 | 217.8 | 213.9 | 215.8 | 2.8 | 1.3% | 450.2 |
| 20000 | 126.6 | 115.2 | 120.9 | 8.0 | 6.9% | 252.2 |
| 10000 | 64.3 | 63.6 | 64.0 | 0.5 | 0.8% | 133.4 |
| 5000 | 37.9 | 36.7 | 37.3 | 0.8 | 2.3% | 77.8 |
| 2500 | 22.2 | 20.9 | 21.6 | 1.0 | 4.7% | 45.0 |
| 1250 | 11.3 | 10.9 | 11.1 | 0.3 | 2.3% | 23.2 |
| 625 | 6.0 | 6.1 | 6.0 | 0.0 | 0.5% | 12.6 |
| 312.5 | 3.5 | 3.3 | 3.4 | 0.1 | 3.7% | 7.2 |
| 0 | 0.5 | 0.5 | 0.5 | 0.0 | 2.1% | 1.0 |

The assay was found to be linear over the selected assay range (312.5 pg/mL to 40,000 pg/mL).

### EXAMPLE 7

### Method According to the Invention Using a Nanoliter-Scale, Microfluidic, Affinity Flow-Through Format with Laser-Induced Fluorescence Detection (Gyros Assay) with Indirect and Direct Alexa Labelling of the Detection-Antibody According to the Invention

Humanized anti CCL2 antibody 11K2 (CCL2-0002) as well as rabbit anti CCL2 antibody 1H11 (CCL2-0011) were labeled with Alexa 647 (Molecular Probes, Invitrogen, Cat A20186). A recombinant cynomolgus CCL2 calibration curve in assay buffer (PBS, 0.1% Tween, 1% BSA) was analyzed with different anti CCL2 capture and detection reagents (1 μg/mL) as described for Example 6.
Anti-CCL2 antibody-Bi (biotin-labeled anti-CCL2 antibody CNT00888), pre-incubated with humanized antibody 11K2 conjugated to digoxygenin and anti-Dig antibody M-1.71.256 (IgG) conjugated to Alexa 647
Anti-CCL2 antibody-Bi (CNT00888-Bi), pre-incubated humanized antibody 11K2 conjugated to Alexa 647
Rabbit anti-CCL2 antibody 2F6 conjugated to biotin (Bi; CCL2-0014), rabbit anti-CCL2 antibody 1H11 conjugated to Alexa 647

| | capture antibody: | | | |
|---|---|---|---|---|
| | CNTO0888-Bi | | CNTO0888-Bi | |
| | detection antibody: | | | |
| | | | pre-incubated humanized | |
| | | | 11K2-Dig + | |
| | humanized | | mAb<Dig>M-1.71.256-IgG- | |
| cyCCL2 | 11K2-Alexa 647 | | Alexa 647 | |
| c [pg/mL] | average | cv | average | cv |
| 51200 | 480.73 | 4.3% | 266.68 | 0.8% |
| 12800 | 200.33 | 0.5% | 86.47 | 3.3% |
| 3200 | 62.07 | 1.1% | 27.75 | 2.9% |
| 800 | 18.02 | 3.4% | 7.86 | 6.9% |
| 200 | 4.92 | 6.5% | 2.38 | 0.7% |
| 50 | 1.27 | 1.6% | 0.88 | 4.3% |
| 12.5 | 0.34 | 5.2% | 0.50 | 3.9% |
| 0 | 0.09 | 27.0% | 0.39 | 6.3% |

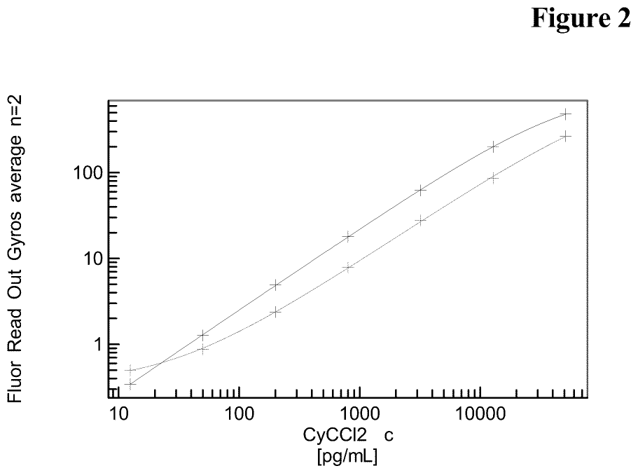

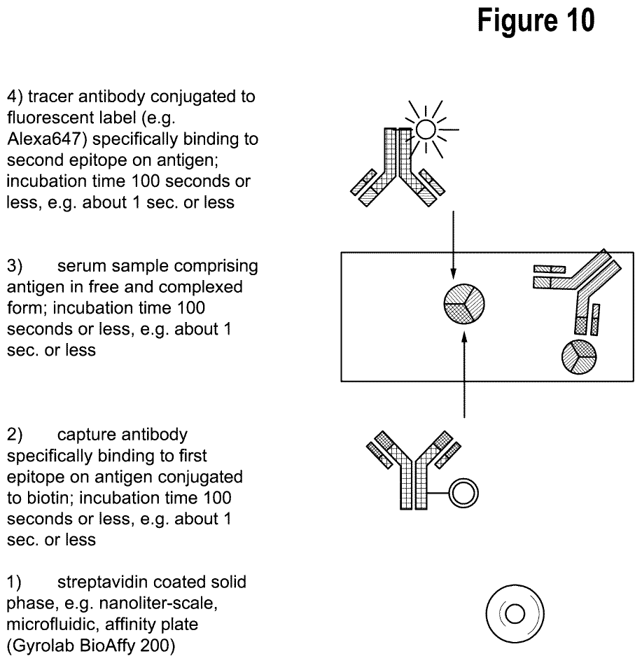

Compared to the pre-incubated detection reagents, sensitivity was increased when directly Alexa 647 labeled detection antibody was used (see Table above and FIGS. 2 and 10).

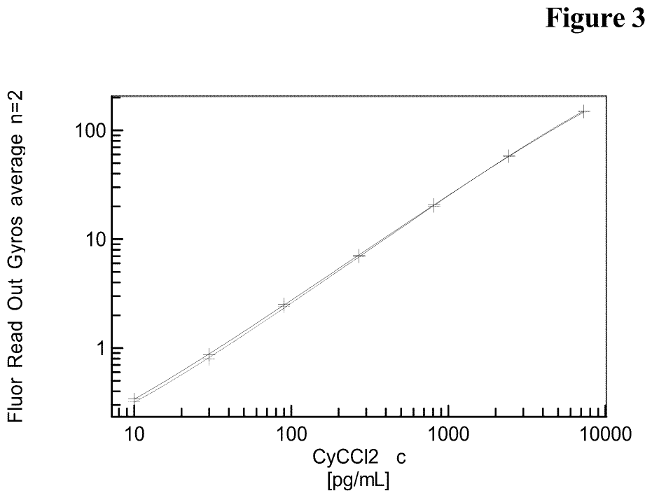

To avoid either false positive results that might occur due to bridging of ADAs directed against constant regions in capture and detection IgGs containing a human backbone as well as false negative results due to neutralizing ADAs directed against the CDRs of the therapeutic molecule that might cross react with the capture and detection reagents, competitive rabbit monoclonal antibodies were used. CCL2 values were back-calculated on the calibration curve and CCL2 recovery (% free) was calculated relative to the non-spiked 5 ng/mL CCL2 value (see also FIG. 3).

| | capture antibody: | | | |
|---|---|---|---|---|
| | CNTO0888-Bi | | 2F6-Bi | |
| | detection antibody: | | | |
| cyCCL2 | humanized 11K2-Alexa 647 | | 1H11-Alexa 647 | |
| c [pg/mL] | average | cv | average | cv |
| 7290 | 147.30 | 4.6% | 151.05 | 1.8% |
| 2430 | 57.60 | 4.1% | 58.58 | 1.2% |
| 810 | 20.75 | 2.6% | 20.08 | 0.2% |
| 270 | 7.10 | 3.4% | 6.88 | 2.9% |
| 90 | 2.54 | 0.6% | 2.42 | 1.8% |
| 30 | 0.87 | 1.6% | 0.80 | 4.4% |
| 10 | 0.34 | 1.3% | 0.32 | 1.4% |
| 0 | 0.06 | 36.3% | 0.04 | 27.7% |

The sensitivity to detect cyCCL2 was comparable between the described human parental capture and detection reagents and the selected competitive rabbit monoclonal anti CCL2 antibodies.

In the POC study four molecules were tested 1: CNTO0888-SG1 (=IgG1 wild type) anti-CCL2 antibody (n=3 animals) as control of maximal total CCL2 accumulation; group 2: a biparatopic anti-CCL2 antibody CKLO2-SG1 (IgG1 wild type) with pH dependent target binding but no Fc-modifications (n=3); group 3: a biparatopic anti-CCL2 antibody CKLO2-SG1100 with pH dependent target binding and Fc-pI and further modifications (n=4) and group 4: biparatopic anti-CCL2 antibody CKLO2-SG1095 with pH dependent target binding, Fc-pI and FcγRII and further modifications (n=4). These four molecules were pre-incubated in assay buffer with 5 ng/mL cyCCL2 for 2 hours in different concentrations and subsequently analyzed in the gyros assay including a cyCCL2 calibration curve. CCL2 values of samples were back-calculated on the calibration curve and recovery values (amount of free CCL2) were calculated relatively to 5 ng/ml CCL2.

| | | CCL2 recovery; free [%] | | | |
|---|---|---|---|---|---|
| pg/mL | ng/ml | CNTO0888- | CKLO2- | CKLO2- | CKLO2- |
| CCL2 | drug | SG1 | SG1 | SG1100 | SG1095 |

capture antibody: CNTO0888-Bi; detection antibody: 11K2-Alexa 647

| | | | | | |
|---|---|---|---|---|---|
| 5000 | 500 | 1.2% | 0.4% | 0.3% | 0.3% |
| 5000 | 250 | 2.1% | 0.7% | 0.5% | 0.6% |
| 5000 | 125 | 4.4% | 2.5% | 1.9% | 2.0% |
| 5000 | 62.5 | 13.3% | 36.1% | 28.8% | 34.3% |
| 5000 | 0 | 96.7% | 104.9% | 99.4% | 106.3% |

capture antibody: 2F6-Bi; detection antibody: 1H11-Alexa 647

| | | | | | |
|---|---|---|---|---|---|
| 5000 | 500 | 1.1% | 0.3% | 0.3% | 0.3% |
| 5000 | 250 | 2.1% | 0.7% | 0.5% | 0.6% |
| 5000 | 125 | 4.7% | 2.5% | 2.0% | 1.9% |
| 5000 | 62.5 | 12.7% | 36.1% | 27.9% | 33.3% |
| 5000 | 0 | 92.7% | 97.9% | 97.7% | 100.9% |

Data depicted in the Table above show comparable results for the set-up with the human parental capture and detection molecules and the competitive rabbit mAbs.

### EXAMPLE 8

### Determination of Free CCL2 in POC Study of CCL2 Sweeping Efficiency in Cynomolgus Monkeys

Free CCL2 serum samples were analyzed with a non-validated, but qualified, Gyrolab™ immunoassay run on a Gyrolab Xplore. A biotinylated anti-CCL2 antibody (M-2F6-IgG) was used as capture reagent and for detection an Alexa 647 labeled anti-CCL2 antibody (M-1H11-IgG) was selected. Both reagents were diluted to 1 μg/mL in PBS, 0.1% Tween, 1% BSA and transferred to a 96-well PCR plate (Fisher Scientific). Cynomolgus monkey CCL2 calibration curve samples, QCs and undiluted serum samples were also transferred to a 96-well PCR plate. Both plates were loaded into the instrument together with a Gyrolab BioAffy 200 nL disc (Gyros Protein Technologies AB). A three-step assay protocol (200-3W-001) was selected. Briefly, the protocol describes the sequential addition of capture reagent, sample and detection reagent to designated streptavidin columns of the Gyrolab BioAffy 200 disc. Each reagent reaches the column at the same time after a short spinning step is applied to the disc. The columns were washed with PBS 0.05% Tween after each step and finally laser induced fluorescence values were recorded within the instrument. The free cynomolgus monkey CCL2 concentration was calculated based on the response of the calibration curve using XL Fit software (IDBS).

To demonstrate assay performance QC samples (High QC 1820 pg/mL cyCCL2, Mid QC 230 pg/mL cyCCL2 and LQC 30 pg/mL CCL2) were prepared in 1× PBS, 0.1% Tween, 1% BSA and analyzed in each run in parallel to cynomolgus pool serum (obtained from biotrend). Calibrators were also prepared in 1× PBS, 0.1% Tween, 1% BSA in the range of 2430 pg/mL to 10 pg/mL. Additionally cynomolgus pooled serum (CPS) was spiked with 7.5 ng/mL and 10 μg/mL CNTO0888 as well as 15 ng/mL and 10 μg/mL CKLO2-SG1095. These samples were also analyzed as QC samples in the respective assay runs (dependent on the group). As shown in the following Table free CCL2 value variation between the assay runs was below 11% for these free QC samples. The assay QCs containing recombinant CCL2 in assay buffer were found to be within +/−20% of the nominal concentration in all 12 assay runs (data not shown).

| | | Measured CCL2 [pg/mL] | | | |
|---|---|---|---|---|---|
| | | CPS with CKLO2-SG1095 | | CPS with CNTO0888 | |
| Run | CPS | 10 μg/mL | 15 ng/ml | 10 μg/mL | 7.5 ng/ml |
| 1 | 586.7 | 13.5 | 128.8 | — | — |
| 2 | 597.3 | 13.3 | 134.1 | — | — |
| 3 | 560.5 | 12.0 | 144.0 | — | — |
| 4 | 570.5 | 14.8 | 116.7 | — | — |
| 5 | 600.5 | 13.4 | 130.6 | — | — |
| 6 | 586.2 | — | — | 15.1 | 272.6 |
| 7 | 593.3 | — | — | 13.7 | 264.0 |
| 8 | 563.0 | — | — | 14.8 | 242.7 |
| 9 | 657.4 | 14.4 | 139.4 | — | — |
| 10 | 568.6 | 17.1 | 142.0 | — | — |
| 11 | 573.0 | 12.2 | 122.1 | — | — |
| 12 | 552.5 | 14.6 | 135.1 | — | — |
| av | 584 | 14 | 133 | 15 | 260 |
| cv | 5% | 11% | 7% | 5% | 6% |

—: not determined

### EXAMPLE 9

### Mouse Study

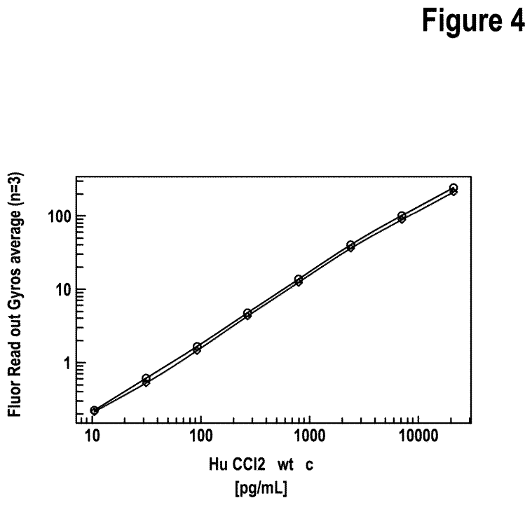

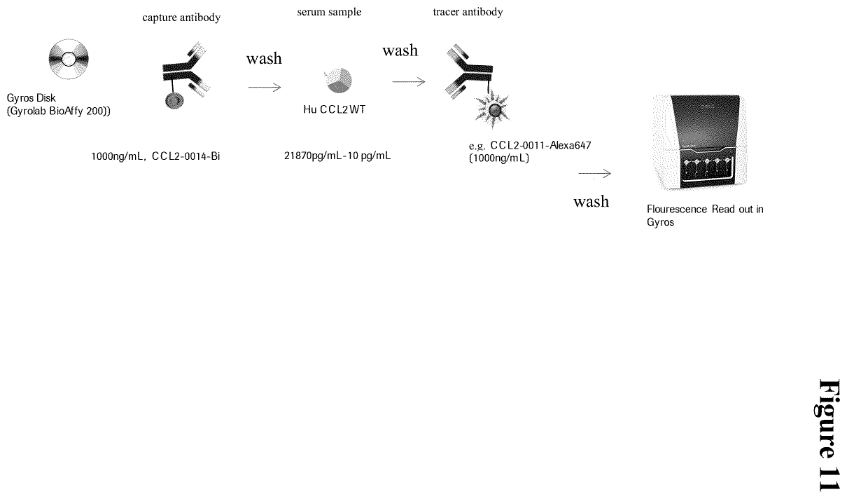

To support studies conducted in B16-huCCL2/CCL2-null mouse models the assay described in Example 8 was performed with recombinant human wild-type CCL2 as calibrator (see FIG. 11 for assay scheme). The assay range was extended in the upper end to 21,870 pg/ml as highest calibrator as huCCL2 values in the transgenic mouse were expected to be higher as in the cynomolgus studies. Linearity of the extended calibration range of two runs is shown in FIG. 4 and in the following Table.

| | Fluor Read | |
|---|---|---|
| Hu wtCCL2 | out Gyros | |
| c [pg/mL] | average (n = 3) | cv in % |
| 21870 | 233.45 | 2.4% |
| 7290 | 98.02 | 8.3% |
| 2430 | 37.35 | 7.5% |
| 810 | 13.46 | 9.5% |
| 270 | 4.72 | 1.0% |
| 90 | 1.65 | 5.5% |
| 30 | 0.61 | 12.3% |
| 10 | 0.23 | 4.5% |
| 0 | 0.06 | 2.6% |

As controls for study pooled mouse serum (MPS) was either spiked with 5 ng/mL recombinant human wild-type CCL2 or with 5 ng/mL recombinant human wild-type CCL2 and 5 μg/mL or 50 ng/mL CKLO2-SG1095. Recovery values were calculated relative to the nominal 5 ng/ml. The corresponding data is shown in the Table below.

| | Measured CCL2 | Recovery |
|---|---|---|
| | [pg/mL] | (%) |
| MPS_5 ng/mL huCCL2 | 5139 | 102.8% |
| MPS_5 ng/mLCCL_5 μg/mLCKLO2- | Below lower limit of | |
| SG1095 | quantification (10 | |
| | pg/mL) | |
| MPS_5 ng/mLCCL_50 ng/mLCKLO2- | 1668 | 33.4% |
| SG1095 | | |

### EXAMPLE 10

### Method According to the Invention for the Determination of Free C5 in 100% Serum Samples

A Gyrolab® workstation was used. 20 μL of test samples, quality control samples, blank samples and each positive control standard were transferred into the designated wells of a multi-well plate. Thereto the respective capture and detection reagent was added. The sealed plate was centrifuged for 10 sec. at at least 3000 g. A 3-step method with two wash solutions for needle washes was used for the analysis in the Gyrolab® workstation. Samples, blanks, quality controls and standards were measured in replicates (N=2).

Result interpretation of samples is based on qualitative interpretation of the FU of the samples and corresponding quality controls (QCs). 1% PMT Fluorescence raw data were exported as Excel File using Gyrolab® Evaluator Software. A standard calibration curve was generated by a non-linear 4-parameter fit using a Wiemer-Rodbard function (e.g. using XLfit for MS Excel): Wiemer Rodbard: [y(x)={(1*A)+((B−A)/{1+{(C/x)AD))))]. A and B are responsible for signal deviation (approximated start and end of the calibration curve). C and D are responsible for the curve shape. Quantitation of the results relative to the positive control antibody is done by back-calculation of the mean signals of the samples using the fitted calibration curve. Representative raw date (fluorescent unit) of the calibration are shown in the following Table.

| Serum | | | | | | | | |
|---|---|---|---|---|---|---|---|---|
| concentration | | | | | | | | |
| [ng/mL] | 3000 | 714 | 170 | 40 | 9.6 | 2.3 | 0.55 | blank |
| Average | 387 | 129 | 30 | 7.2 | 1.7 | 0.38 | 0.08 | 0.02 |
| emission [FU] | | | | | | | | |

1. A method for determining free antigen, that can be specifically bound by an antibody, in an undiluted serum sample comprising free antigen, free antibody and antigen-antibody-complexes, wherein the method comprises the following steps:
a) applying the undiluted serum sample to a solid phase on which a capture antibody has been immobilized to form a capture antibody-antigen complex,
wherein the capture antibody competes with the antibody for binding to a first epitope on the antigen,
b) applying to the solid phase a tracer antibody to form a capture antibody-antigen-tracer antibody complex,
wherein the tracer antibody specifically binds to a second epitope on the antigen,
wherein the epitope of the tracer antibody is not overlapping with the epitope of the capture antibody on the antigen,
c) determining the free antigen of the antibody by determining the tracer antibody in the capture antibody-antigen-tracer antibody complex.

2. The method according to claim 1, wherein in step a) the applying is under conditions that at most 10% of the antibody bound to the antigen are replaced by the capture antibody, wherein in step a) at most 10% of the antibody bound to the antigen is replaced.

3. The method according to claim 1, wherein step a) is
applying the undiluted serum sample to the solid phase on which a capture antibody has been immobilized to form a capture antibody-antigen complex,
wherein the capture antibody competes with the antibody for binding to a first epitope on the antigen,
wherein the sample is incubated with the solid phase for 240 seconds or less.

4. The method according to claim 1, wherein the half-life of the complex of the antigen binding site of the antibody specifically binding to the first epitope on the antigen and the antigen is 100 seconds or less.

5. The method according to claim 1, wherein the antibody is a bispecific antibody; wherein the bispecific antibody comprises a first antigen-binding site that specifically binds to the first epitope on the antigen and a second different antigen-binding site that specifically binds the second epitope on the antigen, wherein the tracer antibody competes with the bispecific antibody for binding to the second epitope on the antigen.

6. The method according to claim 5, wherein the half-life of the complex of the antigen binding site of the bispecific antibody specifically binding to the second epitope on the antigen and the antigen is 20 seconds or less.

7. The method according to claim 1, wherein the capture antibody and the tracer antibody is a non-human, non-humanized antibody.

8. The method according to claim 1, wherein the method is an enzyme-linked immunosorbent assay and the sample is incubated with the solid phase for 180 to 240 seconds.

9. The method according to claim 1, wherein the tracer antibody is incubated with the capture antibody-antigen complex for less than 1200 seconds.

10. The method according to claim 1, wherein the method is a nanoliter-scale, microfluidic, affinity flow-through format with laser-induced fluorescence detection and the sample is incubated with the solid phase for 2 seconds or less.

11. The method according to claim 1, wherein the tracer antibody is incubated with the capture antibody-antigen complex for less than 2 seconds.

12. The method according to claim 1, wherein the antibody is a therapeutic antibody.

13. The method according to claim 1, wherein the method is for determining the amount of free antigen, and step c) is
determining the amount of free antigen of the antibody in the serum sample by determining the amount of the tracer antibody in the capture antibody-antigen-tracer antibody complex.

14. The method according to claim 1, wherein the antigen is human CCL2.

15. The method according to claim 1, wherein the antigen is human C5.


SEQUENCE LISTING (201 sequences)

SEQ ID NO: 1 - protein, 121 aa, synthetic construct
  REGION 1..121: note = CFA0305 VH
QSLEESGGDLVKPGASLAVTCTASGFSSSSSYYMCWVRQAPGKGLEWIGCIYTGSGATYYASWAKGRFTISKTSSTTVTLQMTSLTVADTATYFCASDGGYVTPTHAMYLWGPGTLVTVSS

SEQ ID NO: 2 - protein, 117 aa, synthetic construct
  REGION 1..117: note = CFA0307 VH
QSLEESGGGLVKPGASLTLTCKASGIDFSNFYYICWVRQAPGKGLDLIACIYTVSGYTYYASWAKGRLTISKTSSTTVTLQMTSLTAADTATYFCARDLHAGITNLWGPGTLVTVSS

SEQ ID NO: 3 - protein, 122 aa, synthetic construct
  REGION 1..122: note = CFA0366 VH
QSLEESGGDLVKPGASLSLTCTAAGLDFSSSYYMCWVRQAPGKGLEWIACIYAGSSGIIYYANWAKGRFTISRPSSTTVTLQMTSLTAADTATYFCATYPTYGDGGHAFNLWGPGTLVTVSS

SEQ ID NO: 4 - protein, 117 aa, synthetic construct
  REGION 1..117: note = CFA0501 VH
QSVEESGGRLVTPGTPLTLTCTASGFSLSSYYMNWVRQAPGKGLEWIGMIYGSGATYYASWAKGRFTISKTSSTTVDLKMTSLTTEDTATYFCARQIYSGDNNDNFWGPGTLVTVSS

SEQ ID NO: 5 - protein, 117 aa, synthetic construct; identical to SEQ ID NO: 4
  REGION 1..117: note = CFA0538 VH

SEQ ID NO: 6 - protein, 117 aa, synthetic construct
  REGION 1..117: note = CFA0599 VH
QSVEESGGRLVTPGTPLTLTCTVSGLSLSDNTMTWVRQAPGKGLEWIGIISFGGDAYYASWAKGRFPISKTSTTVDLRITSPTTEDTATYFCARVGAGNIFWYFDLWGPGTLVTVSS

SEQ ID NO: 7 - protein, 116 aa, synthetic construct
  REGION 1..116: note = CFA0666 VH
QSVEESGGRLVTPGTPLTLTCTVSGFSLSTYAMGWVRQAPGKGLEWIGTIDTGDNSFYANWAKGRFTISKTSTTVDLKITSPTAEDTATYFCARNDGSVYNLFNLWGPGTLVTVSS

SEQ ID NO: 8 - protein, 110 aa, synthetic construct
  REGION 1..110: note = CFA0672 VH
QSVEESGGRLVTPGTPLTLTCTASGFSLSGNAINWVRQAPGKGLEWIGCIYTGSDTTYYATWAKGRFTISKTSTTVDLKIASPTTEDTATYFCARGSGLWGPGTLVTVSS

SEQ ID NO: 9 - protein, 116 aa, synthetic construct
  REGION 1..116: note = CFA0675 VH
QSVEESGGRLVTPGTPLTLTCTVSGFSLSTYAMGWVRQAPGKGLEWIGTIDTGDNSFYANWAKGRFTISRTSTTVDLKITSPTAEDTATYFCARNDGSVYNLFNLWGPGTLVTVSS

SEQ ID NO: 10 - protein, 123 aa, synthetic construct
  REGION 1..123: note = 305LO5 VH
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 11 - protein, 110 aa, synthetic construct
  REGION 1..110: note = CFA0305 VL
DVVMTQTPASVSAAVGGTVTIKCQASQNIGSDLAWYQQKPGQPPRLLIYGASKLASGVSSRFSGSGSGKEFTLTISDLECADAVTYYCQCTFVGSSYGNAFGGGTEVVVK

SEQ ID NO: 12 - protein, 110 aa, synthetic construct
  REGION 1..110: note = CFA0307 VL
AIEMTQTPFSVSAAVGGTVTIKCQASQNIYSNLAWYQQKPGQRPKLLIYGASNLESGVPSRFKGSGSGTEYTLTISDLECADAATYYCLQGYSYSNVDDAFGGGTEVVVK

SEQ ID NO: 13 - protein, 112 aa, synthetic construct
  REGION 1..112: note = CFA0366 VL
AQVLTQTPSSVSAAVGGTVTINCQSSQSVYSSDYLSWYQQKPGQPPKLLIYEASKLASGVPPRFSGSGSGTQFTLTISGVQCDDAATYYCQGTYYSSGWYFAFGGGTEVVVK

SEQ ID NO: 14 - protein, 110 aa, synthetic construct
  REGION 1..110: note = CFA0501 VL
AYDMTQTPASVEVAVGGTVTINCQASESISNWLAWYQQKPGQRPKLLIYRASTLASGVSSRFKGSGSGTQFTLTISGVECDDAATYYCQQDYSSSNVDNTFGGGTKVVVE

SEQ ID NO: 15 - protein, 110 aa, synthetic construct
  REGION 1..110: note = CFA0538 VL
AYDMTQTPASVEVAVGGTVTINCQASESISNWLAWYQQKPGQRPKLLIYRASTLASGVSSRFKGSGSGTQFTLTISGVECDDAATYYCQQDYSSSNVDNTFGGGTTVVVE

SEQ ID NO: 16 - protein, 110 aa, synthetic construct
  REGION 1..110: note = CFA0599 VL
AYDMTQTPASVEVSVGGTVTIKCQASESIYSALAWYQQKPGQPPKLLIYSASTLASGVPSRFKGSGSGTEYTLTISDLECADAATYYCQQYYSSTNVHNSFGGGTTVVVE

SEQ ID NO: 17 - protein, 110 aa, synthetic construct
  REGION 1..110: note = CFA0666 VL
DVVMTQTPASVSAAVGGTVTIKCQASENIYSALSWYQQKPGLRPRLLIYYASTLASGVPSRFKGSRSGTEFTLTISDLECADAATYYCQQYYDINSVDNTFGGGTKVVVE

SEQ ID NO: 18 - protein, 111 aa, synthetic construct
  REGION 1..111: note = CFA0672 VL
DVVMTQTPASVSAPVGGTVTIKCQASQNIYSLLAWYQQKPGQPPKLLIYHTSDLASGVPSRFRGSGSGTEFTLTISDLECADAATYYCQCTAYGSSDVGGTFGGGTTVVVE

SEQ ID NO: 19 - protein, 110 aa, synthetic construct
  REGION 1..110: note = CFA0675 VL
DVVMTQTPASVSAAVGGTVTIKCQASENIYSALSWYQQKPGLRPRLLIYYASTLASGVPSRFKGSRSGTEFTLTISDLECADAASYYCQQYYDINSVDNTFGGGTKVVVE

SEQ ID NO: 20 - protein, 110 aa, synthetic construct
  REGION 1..110: note = 305LO5 VL
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 21 - protein, 117 aa, synthetic construct
  REGION 1..117: note = CFA0330 VH
QSLEESGGDLVEPGASLTLTCTASGFSWNSGYDMCWVRQAPGKGLEWIACIYTGSSANTAYANWAKGRFTISKTSSTTVTLQMTGLTAADTATYFCARHDDYFFDLWGPGTLVTVSS

SEQ ID NO: 22 - protein, 115 aa, synthetic construct
  REGION 1..115: note = CFA0341 VH
QSVEESGGRLVTPGTPLTLTCTVSGIDLSSYTMGWVRQAPGKGLECIGYIHSFGSTYYASWAKGRFTISKTSTTVDLEITSPTTEDTATYFCARDVGGSSGWDLWGQGTLVTVSS

SEQ ID NO: 23 - protein, 116 aa, synthetic construct
  REGION 1..116: note = CFA0329 VH
QSLEESGGGLVKPGASLTLTCTASGFSFSSGYYMCWVRQAPGKGLEWIGCIGTISDSTYYASWAKGRFPISKASSTTVTLQMTSLTAADTATYFCARDPYSYGDLWGPGTLVTVSS

SEQ ID NO: 24 - protein, 114 aa, synthetic construct
  REGION 1..114: note = CFA0330 VL
DVVMTQTPASVSESVGGTVTINCQASQSIGNNLAWYQQKPGQPPKLLIYDASNLASGVPSRFKGSRSGTEPTLTNTLTISGVQCADVATYYCQSGWYGNSYVAAFGGGTEVVVK

SEQ ID NO: 25 - protein, 112 aa, synthetic construct
  REGION 1..112: note = CFA0341 VL
DVVMTQTPSSVSAAVGGTVTIKCQASENIYSNLAWYQQKPGQPPKRLIYDASDLASGVPSRFSGSGYGTQFTLTISGVQCEDAATYYCQANYDSTSSSFGNVFGGGTEVVVK

SEQ ID NO: 26 - protein, 110 aa, synthetic construct
  REGION 1..110: note = CFA0329 VL
DLVMTQTPASVSEPVGGTVTIKCQASQSIGTYLAWYQQKPGQPPKLLIYRASTLASGVPSRFKGSGSGTQFTLTISDLECADAATYYCLGGYYGFSYGNAFGGGTEVVVK

SEQ ID NO: 27 - protein, 325 aa, synthetic construct
  REGION 1..325: note = F760G4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 28 - protein, 325 aa, synthetic construct
  REGION 1..325: note = F939G4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLYISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHYHVTQKSLSLSL

SEQ ID NO: 29 - protein, 325 aa, synthetic construct
  REGION 1..325: note = SG402
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 30 - protein, 1676 aa, synthetic construct
  REGION 1..1676: note = human C5
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 31 - protein, 673 aa, synthetic construct
  REGION 1..673: note = human C5, beta chain
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEIL

SEQ ID NO: 32 - protein, 105 aa, synthetic construct
  REGION 1..105: note = human C5, MG1
EQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNG

SEQ ID NO: 33 - protein, 101 aa, synthetic construct
  REGION 1..101: note = human C5, MG2
FLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLP

SEQ ID NO: 34 - protein, 225 aa, synthetic construct
  REGION 1..225: note = human C5, MG1-MG2
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLP

SEQ ID NO: 35 - protein, 1676 aa, synthetic construct
  REGION 1..1676: note = cynomolgus C5
MGLLGILCFLIFLGKTWGQEQTYVISAPKIFRVGASENIVIQVYGYTEAFDATISIKSYPDKKFSYSSGHVHLSSENKFQNSAVLTIQPKQLPGGQNQVSYVYLEVVSKHFSKSKKIPITYDNGFLFIHTDKPVYTPDQSVKVRVYSLNDDLKPAKRETVLTFIDPEGSEIDMVEEIDHIGIISFPDFKIPSNPRYGMWTIQAKYKEDFSTTGTAFFEVKEYVLPHFSVSVEPESNFIGYKNFKNFEITIKARYFYNKVVTEADVYITFGIREDLKDDQKEMMQTAMQNTMLINGIAEVTFDSETAVKELSYYSLEDLNNKYLYIAVTVIESTGGFSEEAEIPGIKYVLSPYKLNLVATPLFLKPGIPYSIKVQVKDALDQLVGGVPVTLNAQTIDVNQETSDLEPRKSVTRVDDGVASFVVNLPSGVTVLEFNVKTDAPDLPDENQAREGYRAIAYSSLSQSYLYIDWTDNHKALLVGEYLNIIVTPKSPYIDKITHYNYLILSKGKIIHFGTREKLSDASYQSINIPVTQNMVPSSRLLVYYIVTGEQTAELVSDSVWLNIEEKCGNQLQVHLSPDADTYSPGQTVSLNMVTGMDSWVALTAVDSAVYGVQRRAKKPLERVFQFLEKSDLGCGAGGGLNNANVFHLAGLTFLTNANADDSQENDEPCKEIIRPRRMLQEKIEEIAAKYKHLVVKKCCYDGVRINHDETCEQRAARISVGPRCVKAFTECCVVASQLRANNSHKDLQLGRLHMKTLLPVSKPEIRSYFPESWLWEVHLVPRRKQLQFALPDSVTTWEIQGVGISNSGICVADTIKAKVFKDVFLEMNIPYSVVRGEQVQLKGTVYNYRTSGMQFCVKMSAVEGICTSESPVIDHQGTKSSKCVRQKVEGSSNHLVTFTVLPLEIGLQNINFSLETSFGKEILVKSLRVVPEGVKRESYSGITLDPRGIYGTISRRKEFPYRIPLDLVPKTEIKRILSVKGLLVGEILSAVLSREGINILTHLPKGSAEAELMSVVPVFYVFHYLETGNHWNIFHSDPLIEKRNLEKKLKEGMVSIMSYRNADYSYSVWKGGSASTWLTAFALRVLGQVHKYVEQNQNSICNSLLWLVENYQLDNGSFKENSQYQPIKLQGTLPVEARENSLYLTAFTVIGIRKAFDICPLVKINTALIKADTFLLENTLPAQSTFTLAISAYALSLGDKTHPQFRSIVSALKREALVKGNPPIYRFWKDSLQHKDSSVPNTGTARMVETTAYALLTSLNLKDINYVNPIIKWLSEEQRYGGGFYSTQDTINAIEGLTEYSLLVKQLRLNMDIDVAYKHKGPLHNYKMTDKNFLGRPVEVLLNDDLVVSTGFGSGLATVHVTTVVHKTSTSEEVCSFYLKIDTQDIEASHYRGYGNSDYKRIVACASYKPSKEESSSGSSHAVMDISLPTGINANEEDLKALVEGVDQLFTDYQIKDGHVILQLNSIPSSDFLCVRFRIFELFEVGFLSPATFTVYEYHRPDKQCTMFYSTSNIKIQKVCEGATCKCIEADCGQMQKELDLTISAETRKQTACNPEIAYAYKVIITSITTENVFVKYKATLLDIYKTGEAVAEKDSEITFIKKVTCTNAELVKGRQYLIMGKEALQIKYNFTFRYIYPLDSLTWIEYWPRDTTCSSCQAFLANLDEFAEDIFLNGC

SEQ ID NO: 36 - protein, 6 aa, synthetic construct
  REGION 1..6: note = CFA0305 HVR-H1
SSYYMC

SEQ ID NO: 37 - protein, 6 aa, synthetic construct
  REGION 1..6: note = CFA0307 HVR-H1
NFYYIC

SEQ ID NO: 38 - protein, 6 aa, synthetic construct
  REGION 1..6: note = CFA0366 HVR-H1
SSYYMC

SEQ ID NO: 39 - protein, 6 aa, synthetic construct
  REGION 1..6: note = CFA0501 HVR-H1
SSYYMN

SEQ ID NO: 40 - protein, 6 aa, synthetic construct
  REGION 1..6: note = CFA0538 HVR-H1
SSYYMN

SEQ ID NO: 41 - protein, 5 aa, synthetic construct
  REGION 1..5: note = CFA0599 HVR-H1
DNTMT

SEQ ID NO: 42 - protein, 5 aa, synthetic construct
  REGION 1..5: note = CFA0666 HVR-H1
TYAMG

SEQ ID NO: 43 - protein, 5 aa, synthetic construct
  REGION 1..5: note = CFA0672 HVR-H1
GNAIN

SEQ ID NO: 44 - protein, 5 aa, synthetic construct
  REGION 1..5: note = CFA0675 HVR-H1
TYAMG

SEQ ID NO: 45 - protein, 6 aa, synthetic construct
  REGION 1..6: note = 305LO5 HVR-H1
SSYYVA

SEQ ID NO: 46 - protein, 17 aa, synthetic construct
  REGION 1..17: note = CFA0305 HVR-H2
CIYTGSGATYYASWAKG

SEQ ID NO: 47 - protein, 17 aa, synthetic construct
  REGION 1..17: note = CFA0307 HVR-H2
CIYTVSGYTYYASWAKG

SEQ ID NO: 48 - protein, 18 aa, synthetic construct
  REGION 1..18: note = CFA0366 HVR-H2
CIYAGSSGIIYYANWAKG

SEQ ID NO: 49 - protein, 16 aa, synthetic construct
  REGION 1..16: note = CFA0501 HVR-H2
MIYGSGATYYASWAKG

SEQ ID NO: 50 - protein, 16 aa, synthetic construct
  REGION 1..16: note = CFA0538 HVR-H2
MIYGSGATYYASWAKG

SEQ ID NO: 51 - protein, 16 aa, synthetic construct
  REGION 1..16: note = CFA0599 HVR-H2
IISFGGDAYYASWAKG

SEQ ID NO: 52 - protein, 16 aa, synthetic construct
  REGION 1..16: note = CFA0666 HVR-H2
TIDTGDNSFYANWAKG

SEQ ID NO: 53 - protein, 17 aa, synthetic construct
  REGION 1..17: note = CFA0672 HVR-H2
CIYTGSDTTYYATWAKG

SEQ ID NO: 54 - protein, 16 aa, synthetic construct
  REGION 1..16: note = CFA0675 HVR-H2
TIDTGDNSFYANWAKG

SEQ ID NO: 55 - protein, 17 aa, synthetic construct
  REGION 1..17: note = 305LO5 HVR-H2
AIYTGSGATYKASWAKG

SEQ ID NO: 56 - protein, 13 aa, synthetic construct
  REGION 1..13: note = CFA0305 HVR-H3
DGGYVTPTHAMYL

SEQ ID NO: 57 - protein, 9 aa, synthetic construct
  REGION 1..9: note = CFA0307 HVR-H3
DLHAGITNL

SEQ ID NO: 58 - protein, 13 aa, synthetic construct
  REGION 1..13: note = CFA0366 HVR-H3
YPTYGDGGHAFNL

SEQ ID NO: 59 - protein, 11 aa, synthetic construct
  REGION 1..11: note = CFA0501 HVR-H3
QIYSGDNNDNF

SEQ ID NO: 60 - protein, 11 aa, synthetic construct
  REGION 1..11: note = CFA0538 HVR-H3
QIYSGDNNDNF

SEQ ID NO: 61 - protein, 12 aa, synthetic construct
  REGION 1..12: note = CFA0599 HVR-H3
VGAGNIFWYFDL

SEQ ID NO: 62 - protein, 11 aa, synthetic construct
  REGION 1..11: note = CFA0666 HVR-H3
NDGSVYNLFNL

SEQ ID NO: 63 - protein, 4 aa, synthetic construct
  REGION 1..4: note = CFA0672 HVR-H3
GSGL

SEQ ID NO: 64 - protein, 11 aa, synthetic construct
  REGION 1..11: note = CFA0675 HVR-H3
NDGSVYNLFNL

SEQ ID NO: 65 - protein, 13 aa, synthetic construct
  REGION 1..13: note = 305LO5 HVR-H3
DGGYDYPTHAMHY

SEQ ID NO: 66 - protein, 11 aa, synthetic construct
  REGION 1..11: note = CFA0305 HVR-L1
QASQNIGSDLA

SEQ ID NO: 67 - protein, 11 aa, synthetic construct
  REGION 1..11: note = CFA0307 HVR-L1
QASQNIYSNLA

SEQ ID NO: 68 - protein, 13 aa, synthetic construct
  REGION 1..13: note = CFA0366 HVR-L1
QSSQSVYSSDYLS

SEQ ID NO: 69 - protein, 11 aa, synthetic construct
  REGION 1..11: note = CFA0501 HVR-L1
QASESISNWLA

SEQ ID NO: 70 - protein, 11 aa, synthetic construct
  REGION 1..11: note = CFA0538 HVR-L1
QASESISNWLA

SEQ ID NO: 71 - protein, 11 aa, synthetic construct
  REGION 1..11: note = CFA0599 HVR-L1
QASESIYSALA

SEQ ID NO: 72 - protein, 11 aa, synthetic construct
  REGION 1..11: note = CFA0666 HVR-L1
QASENIYSALS

SEQ ID NO: 73 - protein, 11 aa, synthetic construct
  REGION 1..11: note = CFA0672 HVR-L1
QASQNIYSLLA

SEQ ID NO: 74 - protein, 11 aa, synthetic construct
  REGION 1..11: note = CFA0675 HVR-L1
QASENIYSALS

SEQ ID NO: 75 - protein, 11 aa, synthetic construct
  REGION 1..11: note = 305LO5 HVR-L1
QASQNIGSSLA

SEQ ID NO: 76 - protein, 7 aa, synthetic construct
  REGION 1..7: note = CFA0305 HVR-L2
GASKLAS

SEQ ID NO: 77 - protein, 7 aa, synthetic construct
  REGION 1..7: note = CFA0307 HVR-L2
GASNLES

SEQ ID NO: 78 - protein, 7 aa, synthetic construct
  REGION 1..7: note = CFA0366 HVR-L2
EASKLAS

SEQ ID NO: 79 - protein, 7 aa, synthetic construct
  REGION 1..7: note = CFA0501 HVR-L2
RASTLAS

SEQ ID NO: 80 - protein, 7 aa, synthetic construct
  REGION 1..7: note = CFA0538 HVR-L2
RASTLAS

SEQ ID NO: 81 - protein, 7 aa, synthetic construct
  REGION 1..7: note = CFA0599 HVR-L2
SASTLAS

SEQ ID NO: 82 - protein, 7 aa, synthetic construct
  REGION 1..7: note = CFA0666 HVR-L2
YASTLAS

SEQ ID NO: 83 - protein, 7 aa, synthetic construct
  REGION 1..7: note = CFA0672 HVR-L2
HTSDLAS

SEQ ID NO: 84 - protein, 7 aa, synthetic construct
  REGION 1..7: note = CFA0675 HVR-L2
YASTLAS

SEQ ID NO: 85 - protein, 7 aa, synthetic construct
  REGION 1..7: note = 305LO5 HVR-L2
GASKTHS

SEQ ID NO: 86 - protein, 12 aa, synthetic construct
  REGION 1..12: note = CFA0305 HVR-L3
QCTFVGSSYGNA

SEQ ID NO: 87 - protein, 12 aa, synthetic construct
  REGION 1..12: note = CFA0307 HVR-L3
LQGYSYSNVDDA

SEQ ID NO: 88 - protein, 12 aa, synthetic construct
  REGION 1..12: note = CFA0366 HVR-L3
QGTYYSSGWYFA

SEQ ID NO: 89 - protein, 12 aa, synthetic construct
  REGION 1..12: note = CFA0501 HVR-L3
QQDYSSSNVDNT

SEQ ID NO: 90 - protein, 12 aa, synthetic construct
  REGION 1..12: note = CFA0538 HVR-L3
QQDYSSSNVDNT

SEQ ID NO: 91 - protein, 12 aa, synthetic construct
  REGION 1..12: note = CFA0599 HVR-L3
QQYYSSTNVHNS

SEQ ID NO: 92 - protein, 12 aa, synthetic construct
  REGION 1..12: note = CFA0666 HVR-L3
QQYYDINSVDNT

SEQ ID NO: 93 - protein, 13 aa, synthetic construct
  REGION 1..13: note = CFA0672 HVR-L3
QCTAYGSSDVGGT

SEQ ID NO: 94 - protein, 12 aa, synthetic construct
  REGION 1..12: note = CFA0675 HVR-L3
QQYYDINSVDNT

SEQ ID NO: 95 - protein, 12 aa, synthetic construct
  REGION 1..12: note = 305LO5 HVR-L3
QSTKVGSSYGNH

SEQ ID NO: 96 - protein, 1680 aa, synthetic construct
  REGION 1..1680: note = mouse C5
MGLWGILCLLIFLDKTWGQEQTYVISAPKILRVGSSENVVIQVHGYTEAFDATLSLKSYPDKKVTFSSGYVNLSPENKFQNAALLTLQPNQVPREESPVSHVYLEVVSKHFSKSKKIPITYNNGILFIHTDKPVYTPDQSVKIRVYSLGDDLKPAKRETVLTFIDPEGSEVDIVEENDYTGIISFPDFKIPSNPKYGVWTIKANYKKDFTTTGTAYFEIKEYVLPRFSVSIELERTFIGYKNFKNFEITVKARYFYNKVVPDAEVYAFFGLREDIKDEEKQMMHKATQAAKLVDGVAQISFDSETAVKELSYNSLEDLNNKYLYIAVTVTESSGGFSEEAEIPGVKYVLSPYTLNLVATPLFVKPGIPFSIKAQVKDSLEQAVGGVPVTLMAQTVDVNQETSDLETKRSITHDTDGVAVFVLNLPSNVTVLKFEIRTDDPELPEENQASKEYEAVAYSSLSQSYIYIAWTENYKPMLVGEYLNIMVTPKSPYIDKITHYNYLILSKGKIVQYGTREKLFSSTYQNINIPVTQNMVPSARLLVYYIVTGEQTAELVADAVWINIEEKCGNQLQVHLSPDEYVYSPGQTVSLDMVTEADSWVALSAVDRAVYKVQGNAKRAMQRVFQALDEKSDLGCGAGGGHDNADVFHLAGLTFLTNANADDSHYRDDSCKEILRSKRNLHLLRQKIEEQAAKYKHSVPKKCCYDGARVNFYETCEERVARVTIGPLCIRAFNECCTIANKIRKESPHKPVQLGRIHIKTLLPVMKADIRSYFPESWLWEIHRVPKRKQLQVTLPDSLTTWEIQGIGISDNGICVADTLKAKVFKEVFLEMNIPYSVVRGEQIQLKGTVYNYMTSGTKFCVKMSAVEGICTSGSSAASLHTSRPSRCVFQRIEGSSSHLVTFTLLPLEIGLHSINFSLETSFGKDILVKTLRVVPEGVKRESYAGVILDPKGIRGIVNRRKEFPYRIPLDLVPKTKVERILSVKGLLVGEFLSTVLSKEGINILTHLPKGSAEAELMSIAPVFYVFHYLEAGNHWNIFYPDTLSKRQSLEKKIKQGVVSVMSYRNADYSYSMWKGASASTWLTAFALRVLGQVAKYVKQDENSICNSLLWLVEKCQLENGSFKENSQYLPIKLQGTLPAEAQEKTLYLTAFSVIGIRKAVDICPTMKIHTALDKADSFLLENTLPSKSTFTLAIVAYALSLGDRTHPRFRLIVSALRKEAFVKGDPPIYRYWRDTLKRPDSSVPSSGTAGMVETTAYALLASLKLKDMNYANPIIKWLSEEQRYGGGFYSTQDTINAIEGLTEYSLLLKQIHLDMDINVAYKHEGDFHKYKVTEKHFLGRPVEVSLNDDLVVSTGYSSGLATVYVKTVVHKISVSEEFCSFYLKIDTQDIEASSHFRLSDSGFKRIIACASYKPSKEESTSGSSHAVMDISLPTGIGANEEDLRALVEGVDQLLTDYQIKDGHVILQLNSIPSRDFLCVRFRIFELFQVGFLNPATFTVYEYHRPDKQCTMIYSISDTRLQKVCEGAACTCVEADCAQLQAEVDLAISADSRKEKACKPETAYAYKVRITSATEENVFVKYTATLLVTYKTGEAADENSEVTFIKKMSCTNANLVKGKQYLIMGKEVLQIKHNFSFKYIYPLDSSTWIEYWPTDTTCPSCQAFVENLNNFAEDLFLNSCE

SEQ ID NO: 97 - protein, 123 aa, synthetic construct [sequence omitted]

SEQ ID NO: 98 - protein, 123 aa, synthetic construct
  REGION 1..123: note = 305LO16 VH
QVQLVESGGGLVQPGRSLRLSCAASGFTVHSSYYMAWVRQAPGKGLEWVGAIFTGSGAEYKAEWVKGRFTISRDNSKNTLYLQMNSLRAEDTAVYYCASDAGYDYPTHAMHYWGRGTLVTVSS

SEQ ID NO: 99 - protein, 123 aa, synthetic construct
  REGION 1..123: note = 305LO18 VH
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGRGTLVTVSS

SEQ ID NO: 100 - protein, 123 aa, synthetic construct
  REGION 1..123: note = 305LO19, 20, 22 VH
QVQLVESGGGLVQPGGSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSS

SEQ ID NO: 101 - protein, 123 aa, synthetic construct
  REGION 1..123: note = 305LO23 VH
QVQLVESGGGLVQPGGSLRLSCAASGFTVHSSYYMAWVRQAPGKGLEWVSGIFTGSGATYKAEWAKGRVTISKDTSKNQVVLTMTNMDPVDTATYYCASDAGYDYPTHAMHYWGQGTLVTVSS

SEQ ID NO: 102 - protein, 110 aa, synthetic construct [sequence omitted]

SEQ ID NO: 103 - protein, 110 aa, synthetic construct
  REGION 1..110: note = 305LO20 VL
DIQMTQSPSSLSASVGDRVTITCRASQGISSSLAWYQQKPGQAPRLLIYGASETESGVPSRFSGSGSGTDFTLTISSLQPEDFATYYCQNTKVGSSYGNTFGGGTKVEIK

SEQ ID NO: 104 - protein, 110 aa, synthetic construct
  REGION 1..110: note = 305LO22, 23 VL
DIQMTQSPSSLSASVGDRVTITCRASQGISSSLAWYQQKPGQAPRLLIYGASTTQSGVPSRFSGSGSGTDFTLTISSLQPEDFATYYCQNTKVGSSYGNTFGGGTKVEIK

SEQ ID NO: 105 - protein, 328 aa, synthetic construct
  REGION 1..328: note = SG115
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 106 - protein, 325 aa, synthetic construct
  REGION 1..325: note = SG422
ASTKGPSVFPLAPCSRSTSGGTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 107 - protein, 325 aa, synthetic construct
  REGION 1..325: note = SG429
ASTKGPSVFPLAPCSRSTSGGTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHAHYTRKELSLSP

SEQ ID NO: 108 - protein, 6 aa, synthetic construct
  REGION 1..6: note = 305LO15, 16, 18, 19, 20, 22, 23 HVR-H1
SSYYMA

SEQ ID NO: 109 - protein, 17 aa, synthetic construct
  REGION 1..17: note = 305LO15, 18, 19, 20, 22 HVR-H2
AIFTGSGAEYKAEWAKG

SEQ ID NO: 110 - protein, 17 aa, synthetic construct
  REGION 1..17: note = 305LO16 HVR-H2
AIFTGSGAEYKAEWVKG

SEQ ID NO: 111 - protein, 17 aa, synthetic construct
  REGION 1..17: note = 305LO23 HVR-H2
GIFTGSGATYKAEWAKG

SEQ ID NO: 112 - protein, 13 aa, synthetic construct
  REGION 1..13: note = 305LO15, 16, 18, 19, 20, 22, 23 HVR-H3
DAGYDYPTHAMHY

SEQ ID NO: 113 - protein, 11 aa, synthetic construct
  REGION 1..11: note = 305LO15, 16, 18, 19, 20, 22, 23 HVR-L1
RASQGISSSLA

SEQ ID NO: 114 - protein, 7 aa, synthetic construct
  REGION 1..7: note = 305LO15, 16, 18, 19, 20 HVR-L2
GASETES

SEQ ID NO: 115 - protein, 7 aa, synthetic construct
  REGION 1..7: note = 305LO22, 23 HVR-L2
GASTTQS

SEQ ID NO: 116 - protein, 12 aa, synthetic construct
  REGION 1..12: note = 305LO15, 16, 18, 19, 20, 22, 23 HVR-L3
QNTKVGSSYGNT

SEQ ID NO: 117 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 118 - skipped

SEQ ID NO: 119 - protein, 18 aa, synthetic construct
  REGION 1..18: note = heavy chain CDR-H2 1A4
CIGAGSSGSTYYASWAKG

SEQ ID NO: 120 - protein, 10 aa, synthetic construct
  REGION 1..10: note = heavy chain CDR-H31A4
TGTEFTYYSL

SEQ ID NO: 121 - protein, 12 aa, synthetic construct
  REGION 1..12: note = light chain CDR-L11A4
QASQSVYNNNMA

SEQ ID NO: 122 - protein, 7 aa, synthetic construct
  REGION 1..7: note = light chain CDR-L21A4
TASSLAS

SEQ ID NO: 123 - protein, 12 aa, synthetic construct
  REGION 1..12: note = light chain CDR-L31A4
AGYKSYSNDEYG

SEQ ID NO: 124 - protein, 6 aa, synthetic construct
  REGION 1..6: note = heavy chain CDR-H11A5
TSYWMC

SEQ ID NO: 125 - protein, 17 aa, synthetic construct
  REGION 1..17: note = heavy chain CDR-H21A5
CISSSIGVTYYASWAEG

SEQ ID NO: 126 - protein, 12 aa, synthetic construct
  REGION 1..12: note = heavy chain CDR-H31A5
TTDDNWNVGFNL

SEQ ID NO: 127 - protein, 12 aa, synthetic construct
  REGION 1..12: note = light chain CDR-L11A5
QASQSIGNRYLS

SEQ ID NO: 128 - protein, 7 aa, synthetic construct
  REGION 1..7: note = light chain CDR-L21A5
GTSTLAS

SEQ ID NO: 129 - protein, 12 aa, synthetic construct
  REGION 1..12: note = light chain CDR-L31A5
QQGATISYLDNA

SEQ ID NO: 130 - protein, 6 aa, synthetic construct
  REGION 1..6: note = heavy chain CDR-H11G9
LYSYMC

SEQ ID NO: 131 - protein, 18 aa, synthetic construct
  REGION 1..18: note = heavy chain CDR-H21G9
CVDAGASGSTYYASWAKG

SEQ ID NO: 132 - protein, 19 aa, synthetic construct
  REGION 1..19: note = heavy chain CDR-H31G9
GILYYTWPYPAGAIDAFDS

SEQ ID NO: 133 - protein, 11 aa, synthetic construct
  REGION 1..11: note = light chain CDR-L11G9
QASESISNYLS

SEQ ID NO: 134 - protein, 7 aa, synthetic construct
  REGION 1..7: note = light chain CDR-L21G9
KASTLAS

SEQ ID NO: 135 - protein, 12 aa, synthetic construct
  REGION 1..12: note = light chain CDR-L31G9
QQSYSSSNVFNT

SEQ ID NO: 136 - protein, 6 aa, synthetic construct
  REGION 1..6: note = heavy chain CDR-H12F6
NNYYMC

SEQ ID NO: 137 - protein, 17 aa, synthetic construct
  REGION 1..17: note = heavy chain CDR-H22F6
CISTDDSNTHYASWAQG

SEQ ID NO: 138 - protein, 13 aa, synthetic construct
  REGION 1..13: note = heavy chain CDR-H32F6
DAHFTSYGYGFDL

SEQ ID NO: 139 - protein, 11 aa, synthetic construct
  REGION 1..11: note = light chain CDR-L12F6
RASEDIENLVA

SEQ ID NO: 140 - protein, 7 aa, synthetic construct
  REGION 1..7: note = light chain CDR-L22F6
QASKLAS

SEQ ID NO: 141 - protein, 12 aa, synthetic construct
  REGION 1..12: note = light chain CDR-L32F6
QGDYGSGWIMYS

SEQ ID NO: 142 - protein, 5 aa, synthetic construct
  REGION 1..5: note = heavy chain CDR-H1CNTO888
SYGIS

SEQ ID NO: 143 - protein, 17 aa, synthetic construct
  REGION 1..17: note = heavy chain CDR-H2CNTO888
GIIPIFGTANYAQKFQG

SEQ ID NO: 144 - protein, 10 aa, synthetic construct
  REGION 1..10: note = heavy chain CDR-H3CNTO888
YDGIYGELDF

SEQ ID NO: 145 - protein, 12 aa, synthetic construct
  REGION 1..12: note = light chain CDR-L1CNTO888
RASQSVSDAYLA

SEQ ID NO: 146 - protein, 7 aa, synthetic construct
  REGION 1..7: note = light chain CDR-L2CNTO888
DASSRAT

SEQ ID NO: 147 - protein, 10 aa, synthetic construct
  REGION 1..10: note = light chain CDR-L3CNTO888
HQYIQLHSFT

SEQ ID NO: 148 - protein, 119 aa, synthetic construct
  REGION 1..119: note = heavy chain variable domain VHCNTO888
QVQLVQSGAEVKKPGSSVKVSCKASGGTFSSYGISWVRQAPGQGLEWMGGIIPIFGTANYAQKFQGRVTITADESTSTAYMELSSLRSEDTAVYYCARYDGIYGELDFWGQGTLVTVSS

SEQ ID NO: 149 - protein, 109 aa, synthetic construct
  REGION 1..109: note = light chain variable domain VLCNTO888
EIVLTQSPATLSLSPGERATLSCRASQSVSDAYLAWYQQKPGQAPRLLIYDASSRATGVPARFSGSGSGTDFTLTISSLEPEDFAVYYCHQYIQLHSFTFGQGTKVEIK

SEQ ID NO: 150 - protein, 5 aa, synthetic construct
  REGION 1..5: note = heavy chain CDR-H1Humanized 11K2 (= 11K2)
DTYMH

SEQ ID NO: 151 - protein, 17 aa, synthetic construct
  REGION 1..17: note = heavy chain CDR-H2Humanized 11K2 (= 11K2)
RIDPANGNTKFDPKFQG

SEQ ID NO: 152 - protein, 8 aa, synthetic construct
  REGION 1..8: note = heavy chain CDR-H3Humanized 11K2 (= 11K2)
GVFGFFDY

SEQ ID NO: 153 - protein, 11 aa, synthetic construct
  REGION 1..11: note = light chain CDR-L1Humanized 11K2 (= 11K2)
KATEDIYNRLA

SEQ ID NO: 154 - protein, 7 aa, synthetic construct
  REGION 1..7: note = light chain CDR-L2Humanized 11K2 (= 11K2)
GATSLET

SEQ ID NO: 155 - protein, 9 aa, synthetic construct
  REGION 1..9: note = light chain CDR-L3Humanized 11K2 (= 11K2)
QQFWSAPYT

SEQ ID NO: 156 - protein, 117 aa, synthetic construct
  REGION 1..117: note = heavy chain variable domain VHHumanized 11K2 (= 11K2)
QVQLVQSGAEVKKPGSSVKVSCKASGLTISDTYMHWVRQAPGQGLEWMGRIDPANGNTKFDPKFQGRVTITADTSTSTAYMELSSLRSEDTAVYYCARGVFGFFDYWGQGTTVTVSS

SEQ ID NO: 157 - protein, 107 aa, synthetic construct
  REGION 1..107: note = light chain variable domain VLHumanized 11K2 (= 11K2)
DIQMTQSPSSLSASVGDRVTITCKATEDIYNRLAWYQQKPGKAPKLLISGATSLETGVPSRFSGSGSGTDYTLTISSLQPEDFATYYCQQFWSAPYTFGGGTKVEIK

SEQ ID NO: 158 - protein, 5 aa, synthetic construct
  REGION 1..5: note = heavy chain CDR-H1ABN912
HYWMS

SEQ ID NO: 159 - protein, 17 aa, synthetic construct
  REGION 1..17: note = heavy chain CDR-H2ABN912
NIEQDGSEKYYVDSVKG

SEQ ID NO: 160 - protein, 13 aa, synthetic construct
  REGION 1..13: note = heavy chain CDR-H3ABN912
DLEGLHGDGYFDL

SEQ ID NO: 161 - protein, 11 aa, synthetic construct
  REGION 1..11: note = light chain CDR-L1ABN912
RASQGVSSALA

SEQ ID NO: 162 - protein, 7 aa, synthetic construct
  REGION 1..7: note = light chain CDR-L2ABN912
DASSLES

SEQ ID NO: 163 - protein, 9 aa, synthetic construct
  REGION 1..9: note = light chain CDR-L3ABN912
QQFNSYPLT

SEQ ID NO: 164 - protein, 6 aa, synthetic construct
  REGION 1..6: note = heavy chain CDR-H1mutated variant CNTO888
  SITE 5: note = MISC_FEATURE - X is I or T
SHYGXS

SEQ ID NO: 165 - protein, 17 aa, synthetic construct
  REGION 1..17: note = heavy chain CDR-H2mutated variant CNTO888
  SITE 2: note = MISC_FEATURE - X is V or I or H
  SITE 4: note = MISC_FEATURE - X is P or H
  SITE 7: note = MISC_FEATURE - X is H or G
GXIXIFXTANYAQKFQG

SEQ ID NO: 166 - protein, 10 aa, synthetic construct
  REGION 1..10: note = heavy chain CDR-H3mutated variant CNTO888
YDAHYGELDF

SEQ ID NO: 167 - protein, 12 aa, synthetic construct
  REGION 1..12: note = light chain CDR-L1mutated variant CNTO888
RASQHVSDAYLA

SEQ ID NO: 168 - protein, 7 aa, synthetic construct
  REGION 1..7: note = light chain CDR-L2mutated variant CNTO888
DASDRAE

SEQ ID NO: 169 - protein, 10 aa, synthetic construct
  REGION 1..10: note = light chain CDR-L3mutated variant CNTO888
HQYIHLHSFT

SEQ ID NO: 170 - protein, 29 aa, synthetic construct
  REGION 1..29: note = heavy chain FR-H1mutated variant CNTO888
QVQLVQSGAEVKKPGSSVKVSCKASGGTF

SEQ ID NO: 171 - protein, 14 aa, synthetic construct
  REGION 1..14: note = heavy chain FR-H2mutated variant CNTO888
WVRQAPGQGLEWMG

SEQ ID NO: 172 - protein, 32 aa, synthetic construct
  REGION 1..32: note = heavy chain FR-H3mutated variant CNTO888
RVTITADESTSTAYMELSSLRSEDTAVYYCAR

SEQ ID NO: 173 - protein, 11 aa, synthetic construct
  REGION 1..11: note = heavy chain FR-H4mutated variant CNTO888
WGQGTLVTVSS

SEQ ID NO: 174 - protein, 23 aa, synthetic construct
  REGION 1..23: note = light chain FR-L1mutated variant CNTO888
EIVLTQSPATLSLSPGERATLSC

SEQ ID NO: 175 - protein, 15 aa, synthetic construct
  REGION 1..15: note = light chain FR-L2mutated variant CNTO888
WYQQKPGQAPRLLIY

SEQ ID NO: 176 - protein, 32 aa, synthetic construct
  REGION 1..32: note = light chain FR-L3mutated variant CNTO888
GVPARFSGSGSGTDFTLTISSLEPEDFAVYYC

SEQ ID NO: 177 - protein, 9 aa, synthetic construct
  REGION 1..9: note = light chain FR-L4mutated variant CNTO888
GQGTKVEIK

SEQ ID NO: 178 - protein, 119 aa, synthetic construct
  REGION 1..119: note = heavy chain variable domain VHmutated variant CNTO888 H0695
QVQLVQSGAEVKKPGSSVKVSCKASGGTFSHYGISWVRQAPGQGLEWMGGVIPIFHTANYAQKFQGRVTITADESTSTAYMELSSLRSEDTAVYYCARYDAHYGELDFWGQGTLVTVSS

SEQ ID NO: 179 - protein, 119 aa, synthetic construct
  REGION 1..119: note = heavy chain variable domain VHmutated variant CNTO888 H0625
QVQLVQSGAEVKKPGSSVKVSCKASGGTFSHYGISWVRQAPGQGLEWMGGIIHIFHTANYAQKFQGRVTITADESTSTAYMELSSLRSEDTAVYYCARYDAHYGELDFWGQGTLVTVSS

SEQ ID NO: 180 - protein, 119 aa, synthetic construct
  REGION 1..119: note = heavy chain variable domain VHmutated variant CNTO888 H0634
QVQLVQSGAEVKKPGSSVKVSCKASGGTFSHYGTSWVRQAPGQGLEWMGGIIHIFGTANYAQKFQGRVTITADESTSTAYMELSSLRSEDTAVYYCARYDAHYGELDFWGQGTLVTVSS

SEQ ID NO: 181 - protein, 119 aa, synthetic construct
  REGION 1..119: note = heavy chain variable domain VHmutated variant CNTO888 H0635
QVQLVQSGAEVKKPGSSVKVSCKASGGTFSHYGISWVRQAPGQGLEWMGGHIHIFGTANYAQKFQGRVTITADESTSTAYMELSSLRSEDTAVYYCARYDAHYGELDFWGQGTLVTVSS

SEQ ID NO: 182 - protein, 109 aa, synthetic construct
  REGION 1..109: note = light chain variable domain VLmutated variant CNTO888 L0616
EIVLTQSPATLSLSPGERATLSCRASQHVSDAYLAWYQQKPGQAPRLLIYDASDRAEGVPARFSGSGSGTDFTLTISSLEPEDFAVYYCHQYIHLHSFTFGQGTKVEIK

SEQ ID NO: 183 - protein, 5 aa, synthetic construct
  REGION 1..5: note = heavy chain CDR-H1mutated variant humanized 11K2
HTYMH

SEQ ID NO: 184 - protein, 17 aa, synthetic construct
  REGION 1..17: note = heavy chain CDR-H2mutated variant humanized 11K2
  SITE 5: note = MISC_FEATURE - X is D or E
RIDPXNHNTKFDPKFQG

SEQ ID NO: 185 - protein, 8 aa, synthetic construct
  REGION 1..8: note = heavy chain CDR-H3mutated variant humanized 11K2
  SITE 7: note = MISC_FEATURE - X is D or E
GVFGFFXH

SEQ ID NO: 186 - protein, 11 aa, synthetic construct
  REGION 1..11: note = light chain CDR-L1mutated variant humanized 11K2
  SITE 3: note = MISC_FEATURE - X is F or T
  SITE 10: note = MISC_FEATURE - X is R or L
KAXEDIYNRXA

SEQ ID NO: 187 - protein, 7 aa, synthetic construct
  REGION 1..7: note = light chain CDR-L2mutated variant humanized 11K2
GATSLEH

SEQ ID NO: 188 - protein, 9 aa, synthetic construct
  REGION 1..9: note = light chain CDR-L3mutated variant humanized 11K2
  SITE 4: note = MISC_FEATURE - X is W or R
QQFXSAPYT

SEQ ID NO: 189 - protein, 30 aa, synthetic construct
  REGION 1..30: note = heavy chain FR-H1mutated variant humanized 11K2
QVQLVQSGAEVKKPGSSVKVSCKASGLTIS

SEQ ID NO: 190 - protein, 14 aa, synthetic construct
  REGION 1..14: note = heavy chain FR-H2mutated variant humanized 11K2
WVRQAPGQGLEWMG

SEQ ID NO: 191 - protein, 32 aa, synthetic construct
  REGION 1..32: note = heavy chain FR-H3mutated variant humanized 11K2
RVTITADTSTSTAYMELSSLRSEDTAVYYCAR

SEQ ID NO: 192 - protein, 11 aa, synthetic construct
  REGION 1..11: note = heavy chain FR-H4mutated variant humanized 11K2
WGQGTTVTVSS

SEQ ID NO: 193 - protein, 23 aa, synthetic construct
  REGION 1..23: note = light chain FR-L1mutated variant 11K2
DIQMTQSPSSLSASVGDRVTITC

SEQ ID NO: 194 - protein, 15 aa, synthetic construct
  REGION 1..15: note = light chain FR-L2mutated variant humanized 11K2
WYQQKPGKAPKLLIH

SEQ ID NO: 195 - protein, 32 aa, synthetic construct
  REGION 1..32: note = light chain FR-L3mutated variant humanized 11K2
GVPSRFSGSGSGTDYTLTISSLQPEDFATYYC

SEQ ID NO: 196 - protein, 10 aa, synthetic construct
  REGION 1..10: note = light chain FR-L4mutated variant humanized 11K2
FGGGTKVEIK

SEQ ID NO: 197 - protein, 117 aa, synthetic construct
  REGION 1..117: note = heavy chain variable domain VHmutated variant humanized 11K2 H1503
QVQLVQSGAEVKKPGSSVKVSCKASGLTISHTYMHWVRQAPGQGLEWMGRIDPDNHNTKFDPKFQGRVTITADTSTSTAYMELSSLRSEDTAVYYCARGVFGFFDHWGQGTTVTVSS

SEQ ID NO: 198 - protein, 117 aa, synthetic construct
  REGION 1..117: note = heavy chain variable domain VHmutated variant humanized 11K2 H1510
QVQLVQSGAEVKKPGSSVKVSCKASGLTISHTYMHWVRQAPGQGLEWMGRIDPDNHNTKFDPKFQGRVTITADTSTSTAYMELSSLRSEDTAVYYCARGVFGFFEHWGQGTTVTVSS

SEQ ID NO: 199 - protein, 117 aa, synthetic construct
  REGION 1..117: note = heavy chain variable domain VHmutated variant humanized 11K2 H1514
QVQLVQSGAEVKKPGSSVKVSCKASGLTISHTYMHWVRQAPGQGLEWMGRIDPENHNTKFDPKFQGRVTITADTSTSTAYMELSSLRSEDTAVYYCARGVFGFFEHWGQGTTVTVSS

SEQ ID NO: 200 - protein, 107 aa, synthetic construct
  REGION 1..107: note = light chain variable domain VLmutated variant humanized 11K2 L1338
DIQMTQSPSSLSASVGDRVTITCKAFEDIYNRRAWYQQKPGKAPKLLIHGATSLEHGVPSRFSGSGSGTDYTLTISSLQPEDFATYYCQQFWSAPYTFGGGTKVEIK

SEQ ID NO: 201 - protein, 107 aa, synthetic construct
  REGION 1..107: note = light chain variable domain VLmutated variant humanized 11K2 L1201
DIQMTQSPSSLSASVGDRVTITCKATEDIYNRLAWYQQKPGKAPKLLIHGATSLEHGVPSRFSGSGSGTDYTLTISSLQPEDFATYYCQQFRSAPYTFGGGTKVEIK